SEQUENCE LISTING (14 sequences)

SEQ ID NO: 1 - DNA, 828 bp, Streptococcus thermophilus
  PTS Mannose/glucose/ fructose subunit IIC gene sequence (manM)
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcctcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttggtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 2 - protein, 275 aa, Streptococcus thermophilus
  PTS Mannose/glucose/ fructose subunit IIC protein sequence (ManM)
MSDMSIISAILVVAVAFLAGLESILDQFQFHQPLVACTLIGAATGNLTAGIMLGGSPQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALGTIGVAIAFIYLNLSKQGGGNGGGNGGGTSSGSGDPIGDILEDY

SEQ ID NO: 3 - DNA, 1167 bp, Streptococcus thermophilus
  Galactokinase gene sequence (galK)
atgaatacatcacagttaagagaaaagtttaaagaagtttttggtgtagaagcagatcatactttcttttcaccaggtcgtattaatttgattggtgagcatacggactacaatggaggtaacgtccttccggtagctattaccctaggtacttacggagcggcccgcaaacgtgatgacaaagttttgcgtttcttctcagctaactttgaagagaagggaatcatcgaagtgccacttgaaaatcttcgttttgaaaaagaacacaactggacaaactatccaaaaggtgttcttcatttcttgcaagaagctgggcatacgattgattcaggtatggatatttacatctatggtaacattccaaacggatcaggcttgtcatcatcatcatctttggaattattgattggtgttattgttgaaaaactttatgaccttaaattggaacgcctggacttggttaaaatcggaaaacaaacggaaaatgactttattggcgttaactctggtatcatggaccaattcgctattggtatgggagctgatcaatgtgcgatttacttggacacaaatactctaaagtatgacttggtaccccttgacctcaaggataatgtcgtagtcatcatgaacactaacaaacgtcgtgaattggctgattctaaatacaatgaacgtcgtgctgaatgtgaaacagcagtatctgaactacaagaaaaattggatatccaaactctcggtgaattagacttcttgacatttgacgcatacagctatttgattaaagatgaaaaccgtatcaaacgtgcacgccatgtagttcttgaaaatcaacgtacacttcaagctcgtaaagctcttgaaacaggagatttggaaggctttggacgccttatgaatgcttctcatgtgtcattggaatatgattacgaagttacaggtcttgaacttgatactttggcacacacagcttgggaacaagaaggagtattaggagcccgcatgacaggagctggtttcggtggatgtgccattgcacttgtaaacaaagacaaagttgaagacttcaaaaaagcagttggtcaacgctatgaagaagtcgttggttatgcaccaagcttctatattgccgaagtaactggtggttcacgagtacttgattaa

SEQ ID NO: 4 - protein, 388 aa, Streptococcus thermophilus
  Galactokinase protein sequence (GalK)
MNTSQLREKFKEVFGVEADHTFFSPGRINLIGEHTDYNGGNVLPVAITLGTYGAARKRDDKVLRFFSANFEEKGIIEVPLENLRFEKEHNWTNYPKGVLHFLQEAGHTIDSGMDIYIYGNIPNGSGLSSSSSLELLIGVIVEKLYDLKLERLDLVKIGKQTENDFIGVNSGIMDQFAIGMGADQCAIYLDTNTLKYDLVPLDLKDNVVVIMNTNKRRELADSKYNERRAECETAVSELQEKLDIQTLGELDFLTFDAYSYLIKDENRIKRARHVVLENQRTLQARKALETGDLEGFGRLMNASHVSLEYDYEVTGLELDTLAHTAWEQEGVLGARMTGAGFGGCAIALVNKDKVEDFKKAVGQRYEEVVGYAPSFYIAEVTGGSRVLD

SEQ ID NO: 5 - DNA, 1700 bp, Streptococcus thermophilus
  Phosphoglucomutase gene sequence (pgm)
atgtcttacactgaaaattatcaaaaatggctcgattttgctgaattgcctgcttatcttcgtgatgagttggtttctatggatgaaaaaactaaagaagatgccttctatactaatcttgaattcggtacagctggtatgcgtggtttaattggcgctggtaccaaccgtattaacatttatgttgttcgtcaagcaaccgaaggtttggcgcaattgatcgactcaaaaggtgaagaagctaaaaaacgtggtgttgctatcgcttatgacagccgtcatttctctcctgagtttgcttttgaatctgcgcaagttttagcagctcatggtattaaatcttatgtgttcgagagccttcgtccaactcctgaattatcattcgcagtacgtcatctccacacatttgctggtatcatgataacggctagccacaacccagctccatttaacggatacaaagtttatggtgaagacggtggacaaatgccacccgctgatgccgatgcattgacagattacatccgtgctatcgataatcctttcactgtcaaattagctgacctcgaagacagcaaggctagcggtctcatcgaaatcatcggtgaaaatgttgatgctgaatacctaaaagaagttaaagacgttaacatcaaccaggacttgattaatgagtatggtcgtgacatgaagattgtatacacttcacttcatggtactggtgaaatgttggttcgccgtgcccttgctcaagctgggtttgatgctgttcaagtcgttgaagctcaagcggttcctcatgctgacttcttaactgttaaatctcctaacccagaaaaccaagatgcctttgctcttgctgaagaactcggtcgtaatgtagatgccgacgtattggttgcaactgaccctgacgcggaccgtcttggcgttgaaatccgccaaccagatggttcatacctcaacctttctggtaaccaaattggtgctatgaagctcacaaaacagctggtactctccctgctaatgctgccctttgtaaatcaatcgtatcaactgaattagttactaagattgcagaaagctacggcgcaacaatgtttaatgtcttgactggcttcaaatttatcggtgaaaagattcatgaatttgaaacacaacacaattacacttacatgtttggttttgaagaaagcttcggttacctcatcaaaccatttgtacgcgataaagacgctatccaagccgttcttatcgttgcagaaattgctgcatactaccgttcacgtggtatgacattggcagatggtatcgaagaaatctacaaacaatatggttacttctcagaaaagacaatttcagttatgctttcaggtgttgatggtgctgcagaaatcaagaaaatcatggacaaattccgtcgcaatgctcctaaacaattcaacaacactgatattgctaaaacagaagacttcttggaacaaacagctactactgctgacggcgtagaaaaattgacaactcctccaagtaacgttttgaaatacatcttggctgatgattcatggtttgccgttcgtccttcaggtacagaaccaaaaatcaaattttacattgcaacagttggagaaactgaagcggatgctaaagaaaaaattgctaacatcgaagcagaaatcaatgcttttgtaggtgaatag

SEQ ID NO: 6 - protein, 591 aa, Streptococcus thermophilus
  phosphoglucomutase protein sequence (Pgm)
MSYTENYQKWLDFAELPAYLRDELVSMDEKTKEDAFYTNLEFGTAGMRGLIGAGTNRINIYVVRQATEGLAQLCATCGCCAAATATATCCTTIDSKGEEAKKRGVAIAYDSRHFSPEFAFESAQVLAAHGIKSYVFESLRPTPELSFAVRHLHTFAGIMITASHNPAPFNGYKVYGEDGGQMPPADADALTDYIRAIDNPFTVKLADLEDSKASGLIEIIGENVDAEYLKEVKDVNINQDLINEYGRDMKIVYTSLHGTGEMLVRRALAQAGFDAVQVVEAQAVPHADFLTVKSPNPENQDAFALAEELGRNVDADVLVATDPDADRLGVEIRQPDGSYLNLSGNQIGAIIAKYILEAHKTAGTLPANAALCKSIVSTELVTKIAESYGATMFNVLTGFKFIGEKIHEFETQHNYTYMFGFEESFGYLIKPFVRDKDAIQAVLIVAEIAAYYRSRGMTLADGIEEIYKQYGYFSEKTISVMLSGVDGAAEIKKIMDKFRRNAPKQFNNTDIAKTEDFLEQTATTADGVEKLTTPPSNVLKYILADDSWFAVRPSGTEPKIKFYIATVGETEADAKEKIANIEAEINAFVGE

SEQ ID NO: 7 - DNA, 1719 bp, Streptococcus thermophilus
  Phosphoglucomutase gene sequence (pgm)
atgtcttacactgaaaattatcaaaaatggctcgattttgctgaattgcctgcttatcttcgtgatgagttggtttctatggatgaaaaaactaaagaagatgccttctatactaatcttgaattcggtacagctggtatgcgtggtttaattggcgctggtaccaaccgtattaacatttatgttgttcgtcaagcaaccgaaggtttggcgcaattgatcgactcaaaaggtgaggaagctaaaaaacgtggtgttgctatcgcttatgacagccgtcatttctctcctgagtttgcttttgaatctgcgcaagttttagcagctcatggtattaaatcttatgtgttcgagagtcttcgtccaactcctgaattatcattcgcagtacgtcatctccacacatttgctggtatcatgataactgctagccacaacccagctccatttaacggatacaaagtttatggtgaagacggtggacaaatgccacccgctgatgccgatgcattgacagattacatccgtgctattgataatccgttcactgtcaaattagctgacctcgaagacagcaaggctagcggtctcatcgaaatcatcggtgaaaatgttgatgctgaatacctaaaagaagttaaagacgttaacatcaaccaggacttgattaatgagtatggtcgtgacatgaagattgtatacacttcacttcatggtactggtgaaatgttggctcgccgtgcccttgctcaagctgggtttgatgctgttcaagtcgttgaagctcaagcggttcctcatgctgacttcttaactgttaaatctcctaacccagaaaaccaagatgcctttgctcttgctgaagaactcggtcgtaatgtagatgccgacgtattggttgcaactgaccctgacgcggaccgtcttggcgttgaaatccgccaaccagatggttcatacctcaacctttctggtaaccaaattggtgctatcatcgccaaatatatccttgaagctcacaaaacagctggtactctccctgctaatgctgccctttgtaaatcaatcgtatcaactgaattagttactaagattgcagaaagctacggtgcaacaatgtttaatgtcttgactggcttcaaatttatcggtgaaaagattcatgaatttgaaacacaacacaattacacttacatgtttggttttgaagaaagcttcggttacctcatcaaaccatttgtacgcgataaagacgctatccaagccgttcttatcgttgcagaaattgctgcatactaccgttcacgtggtatgacattggcagatggtatcgaagaaatctacaaacaatatggttacttctcagaaaagacaatttcagttacgctttcaggtgttgatggtgctgcagaaatcaagaaaatcatggacaaattccgtcgcaatgctcctaaacaattcaacaacactgatattgctaaaacagaagacttcttggaacaaacagctactactgctgacggcgtagaaaaattgacaactcctccaagtaacgttttgaaatacatcttggctgatgattcatggtttgccgttcgtccttcaggtacagaaccaaaaatcaaattttacattgcaacagttggagaaactgaagcggatgctaaagaaaaaattgctaacatcgaagcagaaatcaatgcctttgtaggtgaatag

SEQ ID NO: 8 - protein, 572 aa, Streptococcus thermophilus
  Phosphoglucomutase protein sequence (Pgm)
MSYTENYQKWLDFAELPAYLRDELVSMDEKTKEDAFYTNLEFGTAGMRGLIGAGTNRINIYVVRQATEGLAQLIDSKGEEAKKRGVAIAYDSRHFSPEFAFESAQVLAAHGIKSYVFESLRPTPELSFAVRHLHTFAGIMITASHNPAPFNGYKVYGEDGGQMPPADADALTDYIRAIDNPFTVKLADLEDSKASGLIEIIGENVDAEYLKEVKDVNINQDLINEYGRDMKIVYTSLHGTGEMLARRALAQAGFDAVQVVEAQAVPHADFLTVKSPNPENQDAFALAEELGRNVDADVLVATDPDADRLGVEIRQPDGSYLNLSGNQIGAIIAKYILEAHKTAGTLPANAALCKSIVSTELVTKIAESYGATMFNVLTGFKFIGEKIHEFETQHNYTYMFGFEESFGYLIKPFVRDKDAIQAVLIVAEIAAYYRSRGMTLADGIEEIYKQYGYFSEKTISVTLSGVDGAAEIKKIMDKFRRNAPKQFNNTDIAKTEDFLEQTATTADGVEKLTTPPSNVLKYILADDSWFAVRPSGTEPKIKFYIATVGETEADAKEKIANIEAEINAFVGE

SEQ ID NO: 9 - DNA, 996 bp, Streptococcus thermophilus
  Galactose operon repressor gene sequence (galR)
atggctacattagcagatatcgcaaaattagcaggtgtatctatttcaactgtttcacgtgttcttaataaagatgaaactctttccgtaacagaggatactagacatcggatattaactatcgctgatgaaatcggatatactaaatacaaaacaattaataattcaaaaaaagaaaagtatcaagtggcaattattcaatgggttagtgaagaacacgagttagatgacatctactattataatattagacttggtattgaaaaaagagcctatgaactagactacgagatgcttcactttttcaacgacattccttcaagtctaggagaggaggtcgttggtgtactctgtatcggaaaatttagtcgtgaacaaattgcgaaactagaaagactaaaaaagactctggtctttgttgatagtgatactcttaatcaaggacatccatgtgttactactgattttgaaaactccgtacaatcggcactctgttatcttaaagaacaaggttgtaataatataggcctactcattgggcaagaaaaaacaacagatgcaactgaaatcatctctgatcctcgtttacgttcttatcggaactactgtatggaaaagggaatctatgaccctctttttattctgactggtgacttcactgtccaatctggctatgaacttcttgattctaagattaagagtggagctactttacctgatgcttactttgcggctagtgatagtctagctattggtgcactcagagcacttcaggaaaatggtatcaaggtccctgacgacattcaaattatctcttttaacgatacaactctagctaaacaagtgtatcctccactttctagtgtcactgtctatacagaagaaatgggacgaacagctatggatattctcaataaacaattattagcacctcgaaaaataccaacacttactaaactaggaacaaaattaacattaagaaacagtacaaaatag

SEQ ID NO: 10 - protein, 331 aa, Streptococcus thermophilus
  Galactose operon repressor protein sequence (GalR)
MATLADIAKLAGVSISTVSRVLNKDETLSVTEDTRHRILTIADEIGYTKYKTINNSKKEKYQVAIIQWVSEEHELDDIYYYNIRLGIEKRAYELDYEMLHFFNDIPSSLGEEVVGVLCIGKFSREQIAKLERLKKTLVFVDSDTLNQGHPCVTTDFENSVQSALCYLKEQGCNNIGLLIGQEKTTDATEIISDPRLRSYRNYCMEKGIYDPLFILTGDFTVQSGYELLDSKIKSGATLPDAYFAASDSLAIGALRALQENGIKVPDDIQIISFNDTTLAKQVYPPLSSVTVYTEEMGRTAMDILNKQLLAPRKIPTLTKLGTKLTLRNSTK

SEQ ID NO: 11 - DNA, 969 bp, Streptococcus thermophilus
  Glucose kinase gene sequence (glcK)
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 12 - protein, 322 aa, Streptococcus thermophilus
  Glucose kinase protein sequence (GlcK)
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 13 - DNA, 1719 bp, Streptococcus thermophilus
  Phosphoglucomutase gene sequence (pgm)
atgtcttacactgaaaattatcaaaaatggctcgattttgctgaattgcctgcttatcttcgtgatgagttggtttctatggatgaaaaaactaaagaagatgccttctatactaatcttgaattcggtacagctggtatgcgtggtttaattggcgctggtaccaaccgtattaacatttatgttgttcgtcaagcaaccgaaggtttggcgcaattgatcgactcaaaaggtgaagaagctaaaaaacgtggtgttgctatcgcttatgacagccgtcatttctctcctgagtttgcttttgaatctgcgcaagttttagcagctcatggtattaaatcttatgtgttcgagagccttcgtccaactcctgaattatcattcgcagtacgtcatctccacacatttgctggtatcatgataacggctagccacaacccagctccatttaacggatacaaagtttatggtgaagacggtggacaaatgccacccgctgatgccgatgcattgacagattacatccgtgctatcgataatcctttcactgtcaaattagctgacctcgaagacagcaaggctagcggtctcatcgaaatcatcggtgaaaatgttgatgctgaatacctaaaagaagttaaagacgttaacatcaaccaggacttgattaatgagtatggtcgtgacatgaagattgtatacacttcacttcatggtactggtgaaatgttggttcgccgtgcccttgctcaagctgggtttgatgctgttcaagtcgttgaagctcaagcggttcctcatgctgacttcttaactgttaaatctcctaacccagaaaaccaagatgcctttgctcttgctgaagaactcggtcgtaatgtagatgccgacgtattggttgcaactgaccctgacgcggaccgtcttggcgttgaaatccgccaaccagatggttcatacctcaacctttctggtaaccaaattggtgctatcatcgccaaatatatccttgaagctcacaaaacagctggtactctccctgctaatgctgccctttgtaaatcaatcgtatcaactgaattagttactaagattgcagaaagctacggcgcaacaatgtttaatgtcttgactggcttcaaatttatcggtgaaaagattcatgaatttgaaacacaacacaattacacttacatgtttggttttgaagaaagcttcggttacctcatcaaaccatttgtacgcgataaagacgctatccaagccgttcttatcgttgcagaaattgctgcatactaccgttcacgtggtatgacattggcagatggtatcgaagaaatctacaaacaatatggttacttctcagaaaagacaatttcagttatgctttcaggtgttgatggtgctgcagaaatcaagaaaatcatggacaaattccgtcgcaatgctcctaaacaattcaacaacactgatattgctaaaacagaagacttcttggaacaaacagctactactgctgacggcgtagaaaaattgacaactcctccaagtaacgttttgaaatacatcttggctgatgattcatggtttgccgttcgtccttcaggtacagaaccaaaaatcaaattttacattgcaacagttggagaaactgaagcggatgctaaagaaaaaattgctaacatcgaagcagaaatcaatgcttttgtaggtgaatag

SEQ ID NO: 14 - protein, 572 aa, Streptococcus thermophilus
  Phosphoglucomutase protein sequence (Pgm)
MSYTENYQKWLDFAELPAYLRDELVSMDEKTKEDAFYTNLEFGTAGMRGLIGAGTNRINIYVVRQATEGLAQLIDSKGEEAKKRGVAIAYDSRHFSPEFAFESAQVLAAHGIKSYVFESLRPTPELSFAVRHLHTFAGIMITASHNPAPFNGYKVYGEDGGQMPPADADALTDYIRAIDNPFTVKLADLEDSKASGLIEIIGENVDAEYLKEVKDVNINQDLINEYGRDMKIVYTSLHGTGEMLVRRALAQAGFDAVQVVEAQAVPHADFLTVKSPNPENQDAFALAEELGRNVDADVLVATDPDADRLGVEIRQPDGSYLNLSGNQIGAIIAKYILEAHKTAGTLPANAALCKSIVSTELVTKIAESYGATMFNVLTGFKFIGEKIHEFETQHNYTYMFGFEESFGYLIKPFVRDKDAIQAVLIVAEIAAYYRSRGMTLADGIEEIYKQYGYFSEKTISVMLSGVDGAAEIKKIMDKFRRNAPKQFNNTDIAKTEDFLEQTATTADGVEKLTTPPSNVLKYILADDSWFAVRPSGTEPKIKFYIATVGETEADAKEKIANIEAEINAFVGE

(19) United States
(12) Patent Application Publication
(10) Pub. No.: US 2024/0130384 A1
(43) Pub. Date: Apr. 25, 2024

(54) LACTIC ACID BACTERIA COMPOSITION FOR PREPARING FERMENTED PRODUCTS

(71) Applicant: Chr. Hansen A/S, Hoersholm (DK)

(72) Inventors: Jesper BROEND, Hoersholm (DK); Sonja BLOCH, Hoersholm (DK); Kim Ib SOERENSEN, Hoersholm (DK)

(73) Assignee: Chr. Hansen A/S, Hoersholm (DK)

(21) Appl. No.: 18/546,950

(22) PCT Filed: Feb. 23, 2022
(86) PCT No.: PCT/EP2022/054480

§ 371 Date: Aug. 16, 2023

(30) Foreign Application Priority Data
Feb. 26, 2021 (EP) 21159643.2
Jul. 12, 2021 (EP) 21185317.1

(51) Int. Cl. A23C 9/123; C07K 14/315; C12N 1/20; C12N 9/12; C12N 9/90

(52) U.S. Cl. CPC A23C 9/1238; C07K 14/315; C12N 1/205; C12N 9/1205; C12N 9/90; C12R 2001/46

(57) ABSTRACT

The present invention relates to a composition comprising one or more novel Streptococcus thermophilus strain(s), and the use of said composition for producing a fermented product such as a dairy product with e.g. an increased sweetness. The invention also relates to novel Streptococcus thermophilus strain(s) as such.

### TECHNICAL FIELD OF THE INVENTION

The present invention relates to a composition comprising one or more novel Streptococcus thermophilus strain(s), and the use of said composition for producing a fermented product such as a dairy product with e.g. an increased sweetness. The invention also relates to novel Streptococcus thermophilus strain(s) as such.

### BACKGROUND OF THE INVENTION

Pure fermented milk products are recognized by a tart or sour taste as a result of the conversion of lactose to lactic acid by lactic acid bacteria during fermentation. Such products are, therefore, often sweetened by the addition of fruit, honey, sugar or artificial sweeteners to accommodate the customers' desire for a sweeter tasting.

The food industry has an increasingly high demand for low-calorie sweet-tasting food products in order to help overcome the overweight and obesity problems that have become so prevalent in the last 20 years. Sweetness, usually regarded as a pleasurable sensation, is produced by the presence of sugars and other substances. The perception of sugars is very different. Using sucrose as a 100 reference, the sweetness of lactose is 16, of galactose 32 and of glucose 74 (God-shall (1988). Food Technology 42(II): 71-78). Glucose is thus perceived more than 4 times sweeter than lactose while still having approximately the same level of calories.

Sugar in fermented food products is more often being replaced with sweeteners such as aspartame, acesulfame K, sucralose and saccharin which can provide the sweetness with a lower intake of calories. However, the use of artificial sweeteners may result in an off-taste and several studies indicating that the consumption of artificial sweeteners is connected with drawbacks, such as increasing hunger, allergies, cancer etc., have contributed to consumer's preference for fermented milk products which only contain natural sweeteners or, preferably, contain no added sweetener. Thus, a special challenge lies in developing fermented milk products where the natural (inner) sweetness is high.

The acidity of fermented milk products depends in large part on the lactic acid bacteria present and the process parameters used for preparing the fermented milk product.

Fermentation of the disaccharide lactose is very much studied in lactic acid bacteria because it is the major carbon source in milk. In many species, lactose is cleaved by β-galactosidase into glucose and galactose after uptake. The glucose is phosphorylated by glucokinase to glucose-6-phosphate and fermented via the Embden-Meyerhof-Parnas pathway (glycolysis) by most lactic acid bacteria.

Streptococcus thermophilus (S. thermophilus) is one of the most widely used lactic acid bacteria for commercial thermophilic milk fermentation where the organism is normally used as part of a mixed starter culture, the other component being a Lactobacillus sp., e.g. Lactobacillus delbrueckii subsp. bulgaricus (L. bulgaricus) for yoghurt or Lactobacillus helveticus (L. helveticus) for Swiss-type cheese.

The legal definition of yoghurt in many countries requires S. thermophilus alongside L. bulgaricus. In other countries it requires S. thermophilus alongside L. acidophilus. All species generate desirable amounts of acetaldehyde, an important flavor component in yoghurt.

Lactose and sucrose are fermented more readily by S. thermophilus than their component monosaccharides. In the presence of excess galactose only the glucose portion of the lactose molecule is fermented and galactose accumulates in fermented milk products when S. thermophilus is used. In yoghurt wherein high acid concentrations limit the fermentation, free galactose remains while the free galactose produced in the early stages of Swiss cheese manufacture is later fermented by L. helveticus.

However, galactose fermenting strains of S. thermophilus have been reported by several researchers (Hutkins et al. (1986) J. Dairy Sci. 69(1): 1-8; Vaillancourt et al. (2002) J. Bacteriol. 184(3); 785-793) and in WO2011/026863 (Chr. Hansen) is described a method for obtaining S. thermophilus strains which are galactose fermenting. Several publications from Chr. Hansen have since been published on other galactose fermenting strains.

In order to meet the requirements of the food industry, it has become relevant to propose new strains, in particular S. thermophilus strains, which provide more natural sweetness without extra calories directly into the fermented product (inner sweetness) by excretion of glucose. It has been observed that galactose strains often exhibit a slower acidification profile and it is therefore a need of the industry to provide fast acidifying strains that are either able to ferment galactose or fast acidifying strains that works well with galactose fermenting strains. Fast acidification may shorten the fermentation time and thus improve process economy and/or the interplay between microbial species. The texturizing properties may also be affected in galactose fermenting strains and it is therefore also desired to develop galactose fermenting strains having improved texturizing properties or strains having improved texturizing properties that works well with galactose fermenting strains.

### SUMMARY OF THE INVENTION

The above objects are achieved with the present invention, which among others, is directed to a composition comprising one or more novel galactose fermenting strain(s) of S. thermophilus strain(s). The novel galactose fermenting S. thermophilus strain(s) comprising one or more mutations in the manM gene encoding the glucose/fructose subunit IIC, in the galK gene encoding the galactokinase, in the pgm gene encoding the phosphoglucomutase and/or in the galR gene encoding the galactose. Besides being able to ferment galactose these novel S. thermophilus strain(s) shows fast acidification during fermentation, acidification to a low pH and/or improved texturizing properties when applied alone and/or in combination with other S. thermophilus strain(s) and/or Lactobacillus strain(s).

Thus, in one aspect the present invention relates to a composition comprising a Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(b) the galK gene encoding the galactokinase at a position corresponding to position 139 in SEQ ID NO. 3;
(c) the pgm gene encoding the phosphoglucomutase at a position corresponding to position 490 in SEQ ID NO. 5 or 13;
(d) the pgm gene encoding the phosphoglucomutase at a position corresponding to position 726 in SEQ ID NO. 7;
(e) the galR gene encoding the galactose operon repressor at a position corresponding to position 82 in SEQ ID NO. 9; and
(f) the glcK gene encoding the glucose kinase at a position corresponding to position 805 in SEQ ID NO. 11.

A further aspect of the invention relates to a method of producing a fermented product, comprising fermenting a substrate with a Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(b) the galK gene encoding the galactokinase at a position corresponding to position 139 in SEQ ID NO. 3;
(c) the pgm gene encoding the phosphoglucomutase at a position corresponding to position 490 in SEQ ID NO. 5 or 13;
(d) the pgm gene encoding the phosphoglucomutase at a position corresponding to position 726 in SEQ ID NO. 7;
(e) the galR gene encoding the galactose operon repressor at a position corresponding to position 82 in SEQ ID NO. 9; and
(f) the glcK gene encoding the glucose kinase at a position corresponding to position 805 in SEQ ID NO. 11
   or a composition according to the present invention.

An aspect of the invention therefore relates to a fermented product obtainable by the method of the present invention.

Yet an aspect of the invention relates to a fermented product comprising a Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(b) the galK gene encoding the galactokinase at a position corresponding to position 139 in SEQ ID NO. 3;
(c) the pgm gene encoding the phosphoglucomutase at a position corresponding to position 490 in SEQ ID NO. 5 or 13;
(d) the pgm gene encoding the phosphoglucomutase at a position corresponding to position 726 in SEQ ID NO. 7;
(e) the galR gene encoding the galactose operon repressor at a position corresponding to position 82 in SEQ ID NO. 9; and
(f) the glcK gene encoding the glucose kinase at a position corresponding to position 805 in SEQ ID NO. 11.

A further aspect of the invention relates to the use of at one or more Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(b) the galK gene encoding the galactokinase at a position corresponding to position 139 in SEQ ID NO. 3;
(c) the pgm gene encoding the phosphoglucomutase at a position corresponding to position 490 in SEQ ID NO. 5 or 13;
(d) the pgm gene encoding the phosphoglucomutase at a position corresponding to position 726 in SEQ ID NO. 7;
(e) the galR gene encoding the galactose operon repressor at a position corresponding to position 82 in SEQ ID NO. 9; and
(f) the glcK gene encoding the glucose kinase at a position corresponding to position 805 in SEQ ID NO. 11
   for the manufacture of a fermented product.

Importantly an aspect of the invention relates to a Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(b) the galK gene encoding the galactokinase at a position corresponding to position 139 in SEQ ID NO. 3;
(c) the pgm gene encoding the phosphoglucomutase at a position corresponding to position 490 in SEQ ID NO. 5 or 13;
(d) the pgm gene encoding the phosphoglucomutase at a position corresponding to position 726 in SEQ ID NO. 7;
(e) the galR gene encoding the galactose operon repressor at a position corresponding to position 82 in SEQ ID NO. 9; and
(f) the glcK gene encoding the glucose kinase at a position corresponding to position 805 in SEQ ID NO. 11.

### BRIEF DESCRIPTION OF THE FIGURES

FIG. 1: Milk acidification profiles of DSM 33762 and the mother strain of DSM 33762.

FIG. 2: Milk acidification profile of DSM 33720.

FIG. 3: Adaptive laboratory evolution progress for strain DSM 33719.

FIG. 4: Milk acidification profiles of DSM 33719 and the mother strain of DSM 33719.

### DETAILED DESCRIPTION OF THE INVENTION

Prior to outlining the present invention in more details, a set of terms and conventions is defined:

The term “genus” means genus as defined on the website: www.ncbi.nlm.nih.gov/taxonomy. A bacterial “strain” as used herein refers to a bacterium which remains genetically unchanged when grown or multiplied. A multiplicity of identical bacteria are included.

In the present context, the term “mutant” or “mutant strain” should be understood as a strain derived, or a strain which can be derived, from a strain of the invention (or the mother strain) by means of e.g. genetic engineering, radiation and/or chemical treatment. It is preferred that the mutant is a functionally equivalent mutant, e.g. a mutant that has substantially the same, or improved, properties (e.g. regarding texture, shear stress, viscosity, gel firmness, mouth coating, flavor, post acidification, acidification speed, and/or phage robustness) as the strain from which it is derived. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light, or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the bacterial genome have been shifted with another nucleotide, or deleted, compared to the mother strain. As will be clear to the skilled person mutants of the present invention can also be mother strains.

In the present context, the term “variant” or “variant strain” should be understood as a strain which is functionally equivalent to a strain of the invention, e.g. having substantially the same, or improved, properties or characteristics e.g. texture, acidification speed, viscosity, gel firmness, mouth coating, flavor, post acidification and/or phage robustness). Such variants, which may be identified using appropriate screening techniques, are a part of the present invention.

For purposes of the present invention, the degree of identity between two amino acid sequences is determined using the Needleman-Wunsch algorithm (Needleman and Wunsch (1970) J. Mol. Biol. 48: 443-453) as implemented in the Needle program of the EMBOSS package (EMBOSS: The European Molecular Biology Open Software Suite, Rice et al. (2000) Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labelled “longest identity” (obtained using the—no brief option) is used as the percent identity and is calculated as follows:

(Identical Residues×100)/(Length of Alignment−Total Number of Gaps in Alignment)

In the present description and claims the conventional one-letter and three-letter codes for amino acid residues are used. For ease of reference, amino acid changes in mutants and variants of the invention are described by use of the following nomenclature: amino acid residue in the parent enzyme; position; substituted amino acid residue(s). According to this nomenclature, the substitution of, for instance, an alanine residue for a glycine residue at position 20 is indicated as Ala20Gly or A20G. The deletion of alanine in the same position is shown as Ala20* or A20*. The insertion of an additional amino acid residue (e.g. a glycine) is indicated as Ala20AlaGly or A20AG. The deletion of a consecutive stretch of amino acid residues (e.g. between alanine at position 20 and glycine at position 21) is indicated as DELTA(Ala20-Gly21) or DELTA(A20-G21). When a parent enzyme sequence contains a deletion in comparison to the enzyme sequence used for numbering an insertion in such a position (e.g. an alanine in the deleted position 20) is indicated as *20Ala or *20A. Multiple mutations are separated by a plus sign or a slash. For example, two mutations in positions 20 and 21 substituting alanine and glutamic acid for glycine and serine, respectively, are indicated as A20G+E21S or A20G/E21S. When an amino acid residue at a given position is substituted with two or more alternative amino acid residues these residues are separated by a comma or a slash. For example, substitution of alanine at position 30 with either glycine or glutamic acid is indicated as A20G,E or A20G/E, or A20G, A20E. When a position suitable for modification is identified herein without any specific modification being suggested, it is to be understood that any amino acid residue may be substituted for the amino acid residue present in the position. Thus, for instance, when a modification of an alanine in position 20 is mentioned but not specified, it is to be understood that the alanine may be deleted or substituted for any other amino acid residue (i.e. any one of R, N, D, C, Q, E, G, H, I, L, K, M, F, P, S, T, W, Y, V).

In the context of the present invention, a mutation in the gene (gene mutation) is to be understood as an alteration in the nucleotide sequence of the genome of an organism resulting in changes in the phenotype of said organism, wherein the alteration may be a deletion of a nucleotide, a substitution of a nucleotide by another nucleotide, an insertion of a nucleotide, or a frameshift. In the context of the present invention, a deletion is to be understood as a genetic mutation resulting in the removal of one or more nucleotides of a nucleotide sequence of the genome of an organism; a insertion is to be understood as the addition of one or more nucleotides to the nucleotide sequence; a substitution (or point mutation) is to be understood as a genetic mutation where a nucleotide of a nucleotide sequence is substituted by another nucleotide; a frameshift is to be understood as a genetic mutation caused by a insertion or deletion of a number of nucleotides in a nucleotide sequence that is not divisible by three, therefore changing the reading frame and resulting in a completely different translation from the original reading frame; an introduction of a stop codon is to be understood as a point mutation in the DNA sequence resulting in a premature stop codon; a inhibition of substrate binding of the encoded protein is to be understood as any mutation in the nucleotide sequence that leads to a change in the protein sequence responsible for preventing binding of a substrate to its catalytic site of the protein. Furthermore, a knockout mutant is to be understood as genetic mutation resulting in the removal or deletion of a gene, such as an entire gene or an entire open reading frame from the genome of an organism.

In the present description and claims the conventional one-letter code for nucleotides is used following the analogous principles as described for amino acids nomenclature supra.

Algorithms for aligning sequences and determining the degree of sequence identity between them are well known in the art. For the purpose of the present invention a process may be carried out for aligning nucleotide sequences using blastn as provided by the National Center for Biotechnology Information (NCBI) on https://blast.ncbi.nlm.nih.gov applying standard parameter.

Acidification profile is measured as disclosed in Example 1.

Shear stress is measured as disclosed in Example 4.

In connection with strains of the genus Lactobacillus, the term “CFU” means colony forming units as determined by growth (forming a colony) on an MRS agar plate incubated at anaerobic conditions at 37° C. for 3 days. The MRS agar has the following composition (g/l):
Bacto Proteose Peptone No. 3: 10.0
Bacto Beef extract: 10.0
Bacto Yeast extract: 5.0
Dextrose: 20.0
Sorbitan Monooleate Complex: 1.0
Ammonium Citrate: 2.0
Sodium Acetate: 5.0
Magnesium Sulfate: 0.1
Manganese Sulfate: 0.05
Potassium Phosphate Dibasis: 2.0
Bacto Agar: 15.0
Milli-Q water: 1000 ml.

pH is adjusted to 5.4 or 6.5: pH is adjusted to 6.5 for L. rhamnosus, L. casei and L. paracasei. For all other Lactobacillus species the pH is adjusted to 5.4. In particular, pH is adjusted to 5.4 for L. delbrueckii subsp. bulgaricus; L. acidophilus and L. helveticus. pH is adjusted to 6.5 for L. rhamnosus, L. casei and L. paracasei.

In connection with S. thermophilus, the term “CFU” means colony forming units as determined by growth (forming a colony) on an M 17 agar plate incubated at aerobic conditions at 37° C. for 3 days. The M 17 agar has the following composition (g/l):
Tryptone: 2.5 g
Peptic digest of meat: 2.5 g
Papaic digest of soybean meal: 5.0 g
Yeast extract: 2.5 g
Meat extract: 5.0 g
Lactose: 5.0 g
Sodium-glycero-phosphate: 19.0 g
Magnesium sulphate, 7 H₂O: 0.25 g
Ascorbic acid: 0.5 g
Agar: 15.0 g
Milli-Q water: 1000 ml.
pH is adjusted to final pH 7.1±0.2 (25° C.)

The term “the mutation inactivates the glucokinase protein” as used herein refers to a mutation which results in an “inactivated glucokinase protein”, a glucokinase protein which, if present in a cell, is not able to exert its normal function as well as mutations which prevent the formation of the glucokinase protein or result in degradation of the glucokinase protein. In particular, an inactivated glucokinase protein is a protein which compared to a functional glucokinase protein is not able to facilitate phosphorylation of glucose to glucose-6-phosphate or facilitates phosphorylation of glucose to glucose-6-phosphate at a significantly reduced rate. The gene encoding such an inactivated glucokinase protein compared to the gene encoding a functional glucokinase protein comprises a mutation in the open reading frame (ORF) of the gene, wherein said mutation may include, but is not limited to, a deletion, a frameshift mutation, introduction of a stop codon or a mutation which results in an amino acid substitution, which changes the functional properties of the protein, or a promoter mutation that reduces or abolishes transcription or translation of the gene.

The term “functional glucokinase protein” as used herein refers to a glucokinase protein which, if present in a cell, facilitates phosphorylation of glucose to glucose-6-phosphate.

A “mutant bacterium” or a “mutant strain” as used herein refers to a natural (spontaneous, naturally occurring) mutant bacterium or an induced mutant bacterium comprising one or more mutations in its genome (DNA) which are absent in the wild type DNA. An “induced mutant” is a bacterium where the mutation was induced by human treatment, such as treatment with chemical mutagens, UV- or gamma radiation etc. In contrast, a “spontaneous mutant” or “naturally occurring mutant” has not been mutagenized by man. Mutant bacteria are herein, non-GMO (non-genetically modified organism), i.e. not modified by recombinant DNA technology.

The term “a mutation that reduces the transport of glucose into the cell” as used herein refers to a mutation in a gene encoding a protein involved in transport of glucose which results in an accumulation of glucose in the environment of the cell. The level of glucose in the culture medium of a S. thermophilus strain can readily be measured by methods known to the skilled person.

The term “the mutation inactivates the glucose transporter” as used herein refers to a mutation which results in an “inactivated glucose transporter”, a glucose transporter protein which, if present in a cell, is not able to exert its normal function as well as mutations which prevent the formation of the glucose transporter protein or result in degradation of the glucose transporter protein.

The term “functional glucose transporter protein” as used herein refers to a glucose transporter protein which, if present in a cell, facilitates transport of glucose over a cell membrane.

The term “glucose-deficient” is used in the context of the present invention to characterize lactic acid bacteria (LAB) which either partially or completely have lost the ability to use glucose as a source for cell growth or for maintaining cell viability. A respective deficiency in glucose metabolism can for example be caused by a mutation in a gene inhibiting or inactivating expression or activity of the glucokinase protein and/or the glucose transporter protein responsible for glucose uptake.

LAB with a deficiency in glucose metabolism may increase the glucose concentration in a culture medium, when grown on lactose as carbohydrate source. The increase of glucose is caused by glucose secretion of the glucose-deficient LABs. Increase of glucose concentration in a culture medium can be determined by HPLC analysis, for example using a Dionex CarboPac PA 20 3*150 mm column (Thermo Fisher Scientific, product number 060142).

The term “glucose-positive” is used in the context of the present invention to characterize LAB which either partially or completely have maintained the ability to use glucose as a source for cell growth or maintaining cell viability.

The inventors have surprisingly identified several S. thermophilus strains that fulfil the needs of the industry. One or more of the new galactose fermenting strains show e.g. improved rheological properties (e.g. texture), when applied as part of a mixed culture in a dairy substrate.

Likewise, one or more of the new galactose fermenting strains show e.g. improved acidification properties (e.g. acidification speed), when applied alone or as part of a mixed culture in a dairy substrate (Examples 1-2). Also, one or more of the new galactose fermenting strains is/are capable of acidifying the fermentation media to a lower pH compared to its mother strain this can e.g. be seen in Example 2.

The novel S. thermophilus strains have the capacity to be used in e.g. dairy cultures such as yoghurt cultures to obtain improved sweetness and improved rheological parameters, such as shear stress of the final product. Rheology is closely linked to sensory quality of the product and the interplay between rheology and taste in the final product is therefore of outmost importance. Also, the novel S. thermophilus strains may speed up the fermentation time due to their improved acidification profile.

### Composition

Thus, one aspect of the present invention relates to a composition comprising a Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(a) the galK gene encoding the galactokinase at a position corresponding to position 139 in SEQ ID NO. 3;
(b) the pgm gene encoding the phosphoglucomutase at a position corresponding to position 490 in SEQ ID NO. 5 or 13;
(c) the pgm gene encoding the phosphoglucomutase at a position corresponding to position 726 in SEQ ID NO. 7;
(d) the galR gene encoding the galactose operon repressor at a position corresponding to position 82 in SEQ ID NO. 9; and
(b)the glcK gene encoding the glucose kinase at a position corresponding to position 805 in SEQ ID NO. 11.

In an embodiment the mutation leads to a change in the encoded protein selected from the group consisting of:
a) the PTS Mannose/glucose/fructose subunit IIC at a position corresponding to position 57 in SEQ ID NO 2;
b) the galactokinase at a position corresponding to position 47 in SEQ ID NO 4;
c) the phosphoglucomutase at a position corresponding to position 242 in SEQ ID NO 6 or 14;
d) the phosphoglucomutase at a position corresponding to position 164 in SEQ ID NO 8;
e) the galactose operon repressor at a position corresponding to position 28 in SEQ ID NO 10; and
f) the glucose kinase at a position corresponding to position 268 in SEQ ID NO 12.

If a further embodiment the mutations are:
(a) a substitution from C to T at a position corresponding to position 169 in SEQ ID NO. 1;
(b) a substitution from G to A at a position corresponding to position 139 in SEQ ID NO. 3;
(c) a substitution from A to C at a position corresponding to position 726 in SEQ ID NO. 5 or 13;
(d) a substitution from C to T at a position corresponding to position 490 in SEQ ID NO. 7;
(e) a substitution from C to T at a position corresponding to position 82 in SEQ ID NO. 9; and
(f) a substitution from G to T at a position corresponding to position 805 in SEQ ID NO: 11.

In yet an embodiment the changes in the encoded protein are:
(a) a substitution of Pro to Leu at a position corresponding to position 57 in SEQ ID NO 2;
(b) a substitution of Ile to Val at a position corresponding to position 47 in SEQ ID NO 4;
(c) a substitution of Glu to Asp at a position corresponding to position 242 in SEQ ID NO 6 or 14;
(d) a substitution of Pro to Ser at a position corresponding to position 164 in SEQ ID NO 8;
(e) a substitution of Leu to Phe at a position corresponding to position 28 in SEQ ID NO 10; and
(f) a substitution of Gly to Cys at a position corresponding to position 268 in SEQ ID NO 12.

In a specific embodiment the Streptococcus thermophilus strain comprises the following mutations:
(a) a substitution from C to T at a position corresponding to position 169 in SEQ ID NO.1; a substitution from nucleotide G to A at a position corresponding to position 139 in SEQ ID NO. 3 and a substitution from nucleotide A to C at a position corresponding to position 726 in SEQ ID NO. 5 or 13;
(b) a substitution from C to T at a position corresponding to position 490 in SEQ ID NO. 5 or 13 and a substitution from nucleotide C to T at a position corresponding to position 82 in SEQ ID NO. 7; or
(c) a substitution from G to T at a position corresponding to position 805 in SEQ ID NO: 9.

In terms of the amino acid profile it may be contemplated that the Streptococcus thermophilus strain comprises the following changes in the encoded protein:
(a) a substitution of Pro to Leu at a position corresponding to position 57 in SEQ ID NO 2; a substitution of Ile to Val at a position 47 in SEQ ID NO 4; and a substitution of Glu to Asp at a position corresponding to position 242 in SEQ ID NO 6 or 14;
(b) a substitution of Pro to Ser at a position corresponding to position 164 in SEQ ID NO 8; and a substitution of Leu to Phe at a position corresponding to position 28 in SEQ ID NO 10; or
(c) a substitution of Gly to Cys at a position corresponding to position 268 in SEQ ID NO 12.

In a specific embodiment the S. thermophilus strain is:
(a) DSM 33719 or mutants or variants thereof;
(b) DSM 33720 or mutants or variants thereof; or
(c) DSM 33762 or mutants or variants thereof.

Further specification of the S. thermophilus strain(s) and the mutations disclosed in (a)-(f) above can be found infra i.e. in the part termed “Novel Streptococcus thermophilus mutants”

The composition of the present invention may be provided in several forms. It may be a powder, pellets or tablets. It may be a frozen form, dried form, freeze dried form, or liquid form. Thus, in one embodiment the composition is in frozen, dried, freeze-dried or liquid form.

The composition of the present invention may additionally comprise cryoprotectants, lyoprotectants, antioxidants, nutrients, fillers, flavorants or mixtures thereof. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectantare known to a skilled person in the art. Suitable cryoprotectants or lyoprotectants include mono-, di-, tri-and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate).

In one embodiment, the composition according to the present invention may comprise one or more cryoprotective agent(s) selected from the group consisting of inosine-5′-monophosphate (IMP), adenosine -5′-monophosphate (AMP), guanosine-5′-monophosphate (GMP), uranosine-5′-monophosphate (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

In one embodiment of the invention the cryoprotective agent is an agent or mixture of agents, which in addition to its cryoprotectivity has a booster effect.

The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity e.g. cultures may lose their acid-producing (acidification) activity when kept stored even for shorter periods of time. Thus, viability and booster effect has to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i.e. the production of e.g. lactic acid, acetic acid, formic acid and/or propionic acid, or its metabolite producing activity such as the production of aroma compounds such as acetaldehyde, (a-acetolactate, acetoin, diacetyl and 2,3-butylene glycol (butanediol)).

In one embodiment the composition of the invention contains or comprises from 0.2% to 20% of the cryoprotective agent or mixture of agents measured as % w/w of the material. It is, however, preferable to add the cryoprotective agent or mixture of agents at an amount which is in the range from 0.2% to 15%, from 0.2% to 10%, from 0.5% to 7%, and from 1% to 6% by weight, including within the range from 2% to 5% of the cryoprotective agent or mixture of agents measured as % w/w of the frozen material by weight. In a preferred embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In the present context the term “from x % to y %” means to include the end-points, thus equal to the term “from and including x % to and including y %”

In a further aspect, the composition of the present invention contains or comprises an ammonium salt (e.g. an ammonium salt of organic acid (such as ammonium formate and ammonium citrate) or an ammonium salt of an inorganic acid) as a booster (e.g. growth booster or acidification booster) for bacterial cells, such as cells belonging to the species S. thermophilus, e.g. (substantial) urease negative bacterial cells. The term “ammonium salt”, “ammonium formate”, etc., should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium. In some preferred embodiments, the ammonium source is exogenous. In some particularly preferred embodiments, ammonium is not provided by the dairy substrate. It should of course be understood that ammonia may be added instead of ammonium salt. Thus, the term ammonium salt comprises ammonia (NH₃), NH₄OH, NH₄⁺, and the like.

In one embodiment the composition of the invention may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum

In one embodiment wherein the microorganism produces a polysaccharide (such as EPS) which causes a high/ropy texture in the acidified milk product the acidified milk product is produced substantially free, or completely free of any addition of thickener and/or stabilizer, such as pectin (e.g. HM pectin, LM pectin), gelatin, CMC, Soya Bean Fiber/Soya Bean Polymer, starch, modified starch, carrageenan, alginate, and guar gum. By substantially free should be understood that the product comprises from 0% to 20% (w/w) (e.g. from 0% to 10%, from 0% to 5% or from 0% to 2% or from 0% to 1%) thickener and/or stabilizer.

As disclosed previously the interplay between microbial species is crucial—on only in terms of growth but equally is the rheological properties (such as texture) and the development of taste.

In an important embodiment the composition comprises one or more Streptococcus thermophilus strains selected from the group comprising: DSM 33719, DSM 33719 mutants, DSM 33719 variants, DSM 33720, DSM 33720 mutants, DSM 33720 variants, DSM 33762, DSM 33762 mutants, and DSM 33762 variants.

In a preferred embodiment the composition comprises:
(a) DSM 33720, DSM 33719, and DSM 33762;
(b) DSM 32227 and DSM 33762; or
(c) DSM 33719 and DSM 32227.

As can be seen in Examples 4 and 5 these strain combinations have been tested in terms of texture and acidification profile in fermented products.

The composition may be provided as a mixture or as a kit-of-parts comprising:
(a) a S. thermophilus strain selected from the group consisting of DSM 33720 or mutants or variants thereof, DSM 33719 or mutants or variants thereof, DSM 33762 or mutants or variants thereof, or any combination thereof; and
(b) a strain belonging to the genus Lactobacillus.

Thus, it may be contemplated that the composition may be a mixture or as a kit-of-parts comprising, (a) a S. thermophilus strain comprising the following gene mutations: (i) a substitution of Pro to Leu at a position corresponding to position 57 in SEQ ID NO 2; a substitution of Ile to Val at a position 47 in SEQ ID NO 4 and a substitution of Glu to Asp at a position corresponding to position 242 in SEQ ID NO 6 or 14 and/or (ii) a substitution of Pro to Ser at a position corresponding to position 164 in SEQ ID NO 8 and a substitution of Leu to Phe at a position corresponding to position 28 in SEQ ID NO 10 and/or (iii) a substitution of Gly to Cys at a position corresponding to position 268 in SEQ ID NO 12 and (b) one or more strain belonging to the genus Lactobacillus.

In an embodiment the (i) above is DSM 33762 or mutants or variants thereof, the (ii) is DSM 33720 or mutants or variants thereof and/or the (iii) above is DSM 33719 or mutants or variants thereof.

If applied in e.g. yoghurt production, it may be preferred that the composition further comprises one or more strains belonging to the genus Lactobacillus. The one or more strains of Lactobacillus may be selected from a group consisting of: Lactobacillus delbrueckii, Lactobacillus delbrueckii subsp bulgaricus, Lactobacillus delbrueckii subsp. lactis, Lactobacillus acidophilus, Lacticaseibacillus casei , Lacticaseibacillus paracasei subsp. Paracasei, and Lacticaseibacillus rhamnosus, Limosilactobacillus fermentum, Lactiplantibacillus plantarum subsp. Plantarum and Lactobacillus helveticus.

In a preferred embodiment of the invention the Lactobacillus strain is selected from the group consisting of Lactobacillus delbrueckii subsp. bulgaricus, Lactiplantibacillus plantarum subsp. Plantarum and Lactobacillus acidophilus.

In a particular embodiment the Lactobacillus bacteria strain of the invention is L. bulgaricus. L. bulgaricus is a lactic acid bacterium which is frequently employed for commercial milk fermentation where the organism is normally used as part of a mixed starter culture.

In a specific embodiment the Lactobacillus delbrueckii subsp bulgaricus is DSM 28910 or mutants or variants thereof.

In one embodiment of the invention, the Lactobacillus bacteria strain of the invention is glucose-deficient. In an alternative embodiment of the invention the Lactobacillus bacteria strain of the invention is glucose-positive.

Thus, in a preferred embodiment the composition and/or mixture or kit-of-parts comprises S. thermophilus strains DSM 32227 and/or DSM 33762 and a strain belonging to the species Lactobacillus. Thus, in a preferred embodiment the composition and/or mixture or kit-of-parts comprises S. thermophilus strains DSM 33719 and/or DSM 32227 and a strain belonging to the species Lactobacillus. Thus, in a preferred embodiment the composition and/or mixture or kit-of-parts comprises S. thermophilus strain DSM 33720 and a strain belonging to the species Lactobacillus.

The composition of the present invention may comprise probiotic bacteria. Probiotic bacterial strains may be added before or after fermentation. If added before fermentation the probiotic bacterial strain also acts as fermentative bacteria.

The term “probiotic bacteria” refers to viable bacteria which are administered in adequate amounts to a consumer for the purpose of achieving a health-promoting effect in the consumer. Probiotic bacteria are capable of surviving the conditions of the gastrointestinal tract after ingestion and colonize the intestine of the consumer.

It will be appreciated that the Lactobacillus genus taxonomy was updated in 2020. The new taxonomy is disclosed in Zheng et al. 2020 and will be cohered to herein if not otherwise indicated. For the purpose of the present invention, the table below presents a list of new and old names of some Lactobacillus species relevant to the present invention.

TABLE 1
New and old names of some Lactobacillus species relevant to the present invention.

| Old Name | New Name |
|---|---|
| Lactobacillus reuteri | Limosilactobacillus reuteri |
| Lactobacillus rhamnosus | Lacticaseibacillus rhamnosus |
| Lactobacillus salivarius | Ligilactobacillus salivarius |
| Lactobacillus casei | Lacticaseibacillus casei |
| Lactobacillus paracasei subsp. paracasei | Lacticaseibacillus paracasei subsp. Paracasei |
| Lactobacillus plantarum subsp. plantarum | Lactiplantibacillus plantarum subsp. plantarum |
| Lactobacillus fermentum | Limosilactobacillus fermentum |
| Lactobacillus animalis | Ligilactobacillus animalis |
| Lactobacillus buchneri | Lentilactobacillus buchneri |
| Lactobacillus curvatus | Latilactobacillus curvatus |
| Lactobacillus futsaii | Companilactobacillus futsaii |
| Lactobacillus sakei subsp. sakei | Latilactobacillus sakei subsp. |
| Lactobacillus pentosus | Lactiplantibacillus pentosus |

In a particular embodiment of the invention the probiotic strain according to the present invention is selected from the group consisting of bacteria of the genus Lactobacillus, such as Lactobacillus acidophilus, Lacticaseibacillus paracasei, Lacticaseibacillus rhamnosus, Lacticaseibacillus casei, Lactobacillus delbrueckii, Lactobacillus lactis, Lactiplantibacillus plantarum, Limosilactobacillus reuteri and Lactobacillus johnsonii, the genus Bifidobacterium, such as the Bifidobacterium longum, Bifidobacterium adolescentis, Bifidobacterium bifidum, Bifidobacterium breve, Bifidobacterium animalis subsp. lactis, Bifidobacterium dentium, Bifidobacterium catenulatum, Bifidobacterium angulatum, Bifidobacterium magnum, Bifidobacterium pseudocatenulatum and Bifidobacterium infantis, and the like.

In a particular embodiment of the invention, the probiotic Lactobacillus strain is selected from the group consisting of Lactobacillus acidophilus, Lacticaseibacillus paracasei, Lacticaseibacillus rhamnosus, Lacticaseibacillus casei, Lactobacillus delbrueckii, Lactobacillus lactis, Lactiplantibacillus plantarum, Limosilactobacillus reuteri and Lactobacillus johnsonii.

In a particular embodiment of the invention, the probiotic strain is Lactobacillus acidophilus (LA-5®) deposited as DSM 13241.

In a particular embodiment of the invention, the probiotic Lactobacillus strain is selected from the group consisting of a Lacticaseibacillus rhamnosus strain and a Lacticaseibacillus paracasei strain. In a particular embodiment of the invention, the probiotic strain is Lacticaseibacillus rhamnosus strain LGG® deposited as ATCC 53103. In a particular embodiment of the invention, the probiotic strain is Lacticaseibacillus paracasei strain CRL 431 deposited as ATCC 55544.

In a particular embodiment of the invention, the probiotic Bifidobacterium strain is selected from the group consisting of Bifidobacterium longum, Bifidobacterium adolescentis, Bifidobacterium bifidum, Bifidobacterium breve, Bifidobacterium animalis subsp. lactis, Bifidobacterium dentium, Bifidobacterium catenulatum, Bifidobacterium angulatum, Bifidobacterium magnum, Bifidobacterium pseudocatenulatum and Bifidobacterium infantis. In a particular embodiment of the invention, the probiotic Bifidobacterium probiotic strain is Bifidobacterium animalis subsp. lactis BB-12® deposited as DSM 15954.

The above mixtures or kit-of-parts may be further combined with other lactic acid bacteria such as but not limited to probiotic bacteria. In one embodiment the at one or more lactic acid bacteria is selected from the group consisting of Bifidobacterium such as Bifidobacterium animalis subsp. lactis (e.g.)BB-12®, Lactobacillus acidophilus (LA-5®), Lacticaseibacillus rhamnosus (e.g. LGG®) and any combinations thereof. Which Bifidobacterium, Lactobacillus acidophilus and/or Lacticaseibacillus rhamnosus to apply depend on their application and food to be produced.

In contrast, the expression “A kit-of-part” comprising strain(s) means that strains or culture of strain(s) are physically separated but intended to be used together. Thus, the strains or culture of S. thermophilus strain(s) and Lactobacillus strain(s) are in different boxes or sachets. In an embodiment, the S. thermophilus strain(s) and the Lactobacillus such as e.g. Lactobacillus delbrueckii subsp bulgaricus, Lactobacillus acidophilus, Lacticaseibacillus casei, Lacticaseibacillus paracasei, and/or Limosilactobacillus reuteri strain(s) are under the same format, i.e., are in a frozen format, in the form of pellets or frozen pellets, a powder form, such as a dried or freeze-dried powder.

In a particular embodiment of the present invention, the composition comprises from 10⁴ to 10¹² CFU (colony forming units)/g of the S. thermophilus strain, from 10⁵ to 10¹¹ CFU/g, from 10⁶ to 10¹⁰ CFU/g, or from 10⁷ to 10⁹ CFU/g of the S. thermophilus strain.

In a particular embodiment the composition further comprises from 10⁴ to 10¹² CFU/g of the Lactobacillus strain, from 10⁵ to 10¹¹ CFU/g, from 10⁶ to 10¹⁰ CFU/g, or from 10⁷ to 10⁹ CFU/g of the Lactobacillus strain.

In a particular embodiment the composition comprises from 10⁴ to 10¹² CFU/g, from 10⁵ to 10¹¹ CFU/g, from 10⁶ to 10¹⁰ CFU/g, or from 10⁷ to 10⁹ CFU/g of each of the Lactobacillus delbrueckii subsp bulgaricus, Lactobacillus acidophilus, Lacticaseibacillus casei, Lacticaseibacillus paracasei, and/or Limosilactobacillus reuteri strain(s).

S. thermophilus, Lactobacillus and Lacticaseibacillus such as L. bulgaricus, L. acidophilus, L. casei, L. paracasei, and/or L. rhamnosus and other lactic acid bacteria are commonly used as starter cultures serving a technological purpose in the production of various foods, such as in the dairy industry, such as for fermented milk products. Thus, in another preferred embodiment the composition is suitable as a starter culture.

The composition may be a starter culture such as a yoghurt starter culture.

The composition and/or starter culture may be frozen, spray-dried, freeze-dried, vacuum-dried, air dried, tray dried or in liquid form. Typically, the storage stability of the composition and/or starter culture can be extended by formulating the product with low water activity. By controlling the water activity (Aw), it is possible to predict and regulate the effect of moisture migration on the product. Therefore, it may be preferred that the water activity (Aw) of the dried compositions herein is in the range from 0.01-0.8, preferably in the range from 0.05-0.4.

It will be appreciated that aspects and embodiments disclosed in the parts termed “Method of producing a fermented product” and “Novel Streptococcus thermophilus strains” may be equally relevant to the present part of the specification. Especially the disclosure about the specific strain(s) of the present invention under “Novel Streptococcus thermophilus strains” are applicable to this part of the specification this will also be clear the person skilled in the art.

### Method of Producing a Fermented Product

The term “milk” is to be understood as the lacteal secretion obtained by milking any mammal, such as cows, sheep, goats, buffaloes or camels. In a preferred embodiment, the milk is cow's milk.

The term “milk substrate” may be any raw and/or processed milk material that can be subjected to fermentation according to the method of the invention. Thus, useful milk substrates include, but are not limited to, solutions/suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder or the milk substrate may originate partly from a plant material. Preferably, at least part of the protein in the milk substrate is (i) proteins naturally occurring in mammalian milk, such as casein or whey protein or (ii) proteins naturally occurring in plant milk. However, part of the protein may be proteins which are not naturally occurring in milk.

Prior to fermentation, the milk substrate may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used herein means treatment of the milk substrate to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

Fermentation processes to be used in production of fermented milk products are well known and the person of skill in the art will know how to select suitable process conditions, such as temperature, oxygen, amount and characteristics of microorganism(s) and process time. Obviously, fermentation conditions are selected so as to support the achievement of the present invention, i.e. to obtain a fermented product such as a dairy or dairy analogue product in solid or liquid form (fermented milk product).

The term “fermented milk product” as used herein refers to a food or feed product wherein the preparation of the food or feed product involves fermentation of a milk substrate with lactic acid bacteria. “Fermented milk product” as used herein includes but is not limited to products such as yogurt and cheese. Examples of cheeses which are prepared by fermentation with S. thermophilus and Lactobacillus delbrueckii subsp. bulgaricus include Mozzarella and pizza cheese (Hoier et al. (2010) in The Technology of Cheesemaking, 2^{nd} Ed. Blackwell Publishing, Oxford; 166-192). Preferably, the fermented milk product is a yogurt.

In the present context the term “starter culture” is a culture which is a preparation (composition) of one or more bacterial strains (such as lactic acid bacteria strains) to assist the beginning of the fermentation process in preparation of fermented products such as various foods, feeds and beverages.

In the present context, a “yoghurt starter culture” is a bacterial culture which comprises one or more Lactobacillus selected from a L. bulgaricus strain and/or an L. acidophilus strain and one or more S. thermophilus strains. In accordance herewith, a “yoghurt” refers to a fermented milk product obtainable by inoculating and fermenting a milk substrate with a composition comprising a Lactobacillus strain such as L. bulgaricus and/or L. acidophilus and a S. thermophilus strain.

A further aspect of the present invention relates to a method of producing a fermented product, comprising fermenting a substrate with a Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(b) the galK gene encoding the galactokinase at a position corresponding to position 139 in SEQ ID NO. 3;
(c) the pgm gene encoding the phosphoglucomutase at a position corresponding to position 490 in SEQ ID NO. 5 or 13;
(d) the pgm gene encoding the phosphoglucomutase at a position corresponding to position 726 in SEQ ID NO. 7;
(e) the galR gene encoding the galactose operon repressor at a position corresponding to position 82 in SEQ ID NO. 9; and
(f) the glcK gene encoding the glucose kinase at a position corresponding to position 805 in SEQ ID NO. 11
   or a composition of the present invention.

It may be preferred that the substrate is a milk substrate. The milk substrate may be an animal derived substrate however, the milk substrate need not be purely animal derived, it may further comprise a plant derived substrate. The fermented product may be a food product which again may be a dairy product.

Depending on the product to be produced the substrate may be a milk substrate. A milk substrate is particularly preferred when fermented milk products such as yoghurt, buttermilk or kefir is the final product.

The milk substrate may be an animal or plant derived product. Thus, in an embodiment the fermented product is a food product such as a dairy product. The dairy product may be selected from the group consisting of a fermented milk product such as but not limited to yoghurt, buttermilk and kefir or cheese such as but not limited to fresh cheese or pasta filata.

Even though the fermented product and/or the food product itself comprise acid and flavor generated during fermentation it may be desired that fermented product and/or the dairy product comprises an ingredient selected from the group consisting of a fruit concentrate, a syrup, a probiotic bacterial strain or culture, a coloring agent, a thickening agent, a flavoring agent, a preserving agent and mixtures thereof.

Likewise, an enzyme may be added to the substrate e.g. the milk substrate before, during and/or after the fermenting, the enzyme being selected from the group consisting of an enzyme able to crosslink proteins, transglutaminase, an aspartic protease, lactase, chymosin, rennet and mixtures thereof.

In one embodiment the fermented product may be in the form of a stirred type product, a set type product or a drinkable product.

Clearly another aspect of the present invention relates to a fermented product obtainable by the method of the present invention. An aspect of the present invention is therefore also a fermented product comprising a Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(b) the galK gene encoding the galactokinase at a position corresponding to position 139 in SEQ ID NO. 3;
(c) the pgm gene encoding the phosphoglucomutase at a position corresponding to position 490 in SEQ ID NO. 5 or 13;
(d) the pgm gene encoding the phosphoglucomutase at a position corresponding to position 726 in SEQ ID NO. 7;
(e) the galR gene encoding the galactose operon repressor at a position corresponding to position 82 in SEQ ID NO. 9; and
(f) the glcK gene encoding the glucose kinase at a position corresponding to position 805 in SEQ ID NO. 11.

A further aspect of the present invention relates to the use of a Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(b) the galK gene encoding the galactokinase at a position corresponding to position 139 in SEQ ID NO. 3;
(c) the pgm gene encoding the phosphoglucomutase at a position corresponding to position 490 in SEQ ID NO. 5 or 13;
(d) the pgm gene encoding the phosphoglucomutase at a position corresponding to position 726 in SEQ ID NO. 7;
(e) the galR gene encoding the galactose operon repressor at a position corresponding to position 82 in SEQ ID NO. 9; and
(f) the glcK gene encoding the glucose kinase at a position corresponding to position 805 in SEQ ID NO. 11 for the manufacture of a fermented product. Again, the fermented product may be a food product such as a dairy or dairy analogue product.

It will be appreciated that aspects and embodiments disclosed in the parts termed “Composition” and “Novel Streptococcus thermophilus strain(s)” may be equally relevant to the present part of the specification. Especially the disclosure about the specific strain(s) of the present invention under “Novel Streptococcus thermophilus strain(s)” are applicable to this part of the specification this will also be clear the person skilled in the art.

Novel Streptococcus thermophilus Strains

The present inventors surprisingly discovered a Streptococcus thermophilus strain having a mutation in one or more genes selected from the group consisting of:
(a) the manM gene encoding the PTS Mannose/glucose/fructose subunit IIC at a position corresponding to position 169 in SEQ ID NO. 1;
(b) the galK gene encoding the galactokinase at a position corresponding to position 139 in SEQ ID NO. 3;
(c) the pgm gene encoding the phosphoglucomutase at a position corresponding to position 490 in SEQ ID NO. 5 or 13;
(d) the pgm gene encoding the phosphoglucomutase at a position corresponding to position 726 in SEQ ID NO. 7;
(e) the galR gene encoding the galactose operon repressor at a position corresponding to position 82 in SEQ ID NO. 9; and
(f) the glcK gene encoding the glucose kinase at a position corresponding to position 805 in SEQ ID NO. 11.

It may be contemplated that the S. thermophilus strain comprises a nucleotide sequence which is at least 50% identical to SEQ ID NO: 1, 3, 5, 7, 11 and/or 13. Preferably, the S. thermophilus strain comprises a nucleotide sequence which is at least 60%, at least 70%, at least 80%, at least 90%, at least 95%, at least 96%, at least 97%, at least 98% or at least 99% identical to SEQ ID NO: 1, 3, 5, 7, 11 and/or 13.

Accordingly, on the amino acid level, it may be further contemplated that the mutation leads to a change in the encoded protein selected from the group consisting of:
a) the PTS Mannose/glucose/fructose subunit IIC at a position corresponding to position 57 in SEQ ID NO 2;
b) the galactokinase at a position corresponding to position 47 in SEQ ID NO 4;
c) the phosphoglucomutase at a position corresponding to position 242 in SEQ ID NO 6 or 14;
d) the phosphoglucomutase at a position corresponding to position 164 in SEQ ID NO 8;
e) the galactose operon repressor at a position corresponding to position 28 in SEQ ID NO 10; and
f) the glucose kinase at a position corresponding to position 268 in SEQ ID NO 12.

The galK gene encoding galactokinase that is an enzyme of the Leloir pathway for galactose metabolism and converts α-galactose to galactose-1-phosphate.

The pgm gene encoding the phosphoglucomutase that is an enzyme that convert the reversible reaction between β-glucose-1-phosphate (G1P) to glucose-6-phosphate (G6P)

The galR gene encoding the galactose operon repressor that regulates the galactose operon of Streptococcus at the transcriptional level.

In a particular embodiment of the use of the invention, the S. thermophilus strain is galactose-fermenting and carries a mutation in the DNA sequence of the glcK gene (more specifically in a position corresponding to position 805 in SEQ ID NO. 11), encoding a glucokinase protein, wherein the mutation inactivates the glucokinase protein or has a negative effect on expression of the gene.

In preferred embodiments the mutation reduces the activity (the rate of phosphorylation of glucose to glucose-6-phosphate) of the glucokinase protein with at least 50%, at least 60%, at least 70%, at least 80%, or at least 90%.

The glucokinase activity can be determined by the glucokinase enzymatic assays as described by Pool et a/. (2006. Metabolic Engineering 8;456-464).

In a particular embodiment of the use of the invention, the S. thermophilus strain carries a mutation that reduces the transport of glucose into the cell. In a specific embodiment, the S. thermophilus strain carries a mutation in a gene encoding a component of a glucose transporter, wherein the mutation inactivates the glucose transporter or has a negative effect on expression of the gene.

In a more specific embodiment, the S. thermophilus strain carries a mutation in the DNA sequence of the manM gene encoding the PTS Mannose/glucose/fructose subunit IIC (may also be termed IIC^{Man} protein of the glucose/mannose phosphotransferase system and the two is therefor used herein interchangeably), wherein the mutation inactivates the IIC^{Man} protein or has a negative effect on expression of the gene. In preferred embodiments the mutation reduces the transport of glucose into the cell with at least 50%, at least 60%, at least 70%, at least 80%, or at least 90% when compared to a cell without such mutation.

The transport of glucose into the cell can be determined by the glucose uptake assay as described by Cochu et al. (2003) Appl Environ Microbiol 69(9); 5423-5432).

Preferably, the S. thermophilus strain carries a mutation in a gene encoding a component of a glucose transporter, wherein the mutation inactivates the glucose transporter protein or has a negative effect on expression of the gene.

The component may be any component of a glucose transporter protein which is critical for the transport of glucose. E.g. it is contemplated that inactivation of any component of the glucose/mannose phosphotransferase system in S. thermophilus will result in inactivation of the glucose transporter function.

In particular, an inactivated glucose transporter protein is a protein which compared to a functional glucose transporter protein is not able to facilitate transport of glucose over a plasma membrane or facilitates transport of glucose over a plasma membrane at a significantly reduced rate. The gene encoding such an inactivated glucose transporter protein compared to the gene encoding a functional glucose transporter protein comprises a mutation in the open reading frame (ORF) of the gene, wherein said mutation may include, but is not limited to, a deletion, a frameshift mutation, introduction of a stop codon or a mutation which results in an amino acid substitution, which changes the functional properties of the protein, or a promoter mutation that reduces or abolishes transcription or translation of the gene.

In preferred embodiments the mutation reduces the activity (the rate of transport of glucose) of the glucose transporter protein by at least 50%, at least 60%, at least 70%, at least 80%, at least 90%. The glucose transporter activity can be determined by the glucose uptake assay as described by Cochu et al. (2003. Appl Environ Microbiol 69(9); 5423-5432).

In a particular embodiment the S. thermophilus strain may increases the amount of glucose in 9.5% B-milk to at least 5 mg/ml when inoculated into the 9.5% B-milk at a concentration of 1.0E06-1.0E07 CFU/ml and grown at 40° C. for 20 hours.

In a particular embodiment of the use of the invention, the S. thermophilus strain DSM 33719 and/or DSM 33762 may increases the amount of glucose in 9.5% B-milk with 0.05% sucrose to at least 5 mg/ml when inoculated into the 9.5% B-milk with 0.05% sucrose at a concentration of 1.0E06-1.0E07 CFU/ml and grown at 40° C. for 20 hours.

In the present context, 9.5% B-milk is heat-treated milk made with reconstituted low fat skim milk powder to a level of dry matter of 9.5% and pasteurized at 99° C. for 30 min. followed by cooling to 40° C.

In more preferred embodiments of the invention the mutant strain leads to an increase in the amount of glucose to at least 4 mg/ml, at least 5 mg/ml, at least 6 mg/ml, at least 7 mg/ml, at least 8 mg/ml, at least 9 mg/ml, at least 10 mg/ml, at least 11 mg/ml, at least 12 mg/ml, at least 13 mg/ml, at least 14 mg/ml, at least 15 mg/ml, at least 20 mg/ml, or at least 25 mg/ml.

In a further embodiment the S. thermophilus strain DSM 33719 and/or DSM 33762 may increase the amount of glucose in a milk base comprising 0.1% sucrose, 4% protein (adjusted with skimmed milk powder) and 1.5% fat (1.5% fat milk adjusted with 9% cream) to at least 5 mg/ml when inoculated together with one or more further lactic acid bacteria strain at a concentration of 1.0E06 -1.0E08 CFU/ml and grown at 40° C. for 20 hours. In more preferred embodiments of the invention the amount of glucose is increased to at least 4 mg/ml, at least 5 mg/ml, at least 6 mg/mL, at least 7 mg/mL, at least 8 mg/mL, at least 9 mg/ml, at least 10 mg/ml, at least 11 mg/ml, at least 12 mg/ml, at least 13 mg/ml, at least 14 mg/ml, at least 15 mg/ml, at least 20 mg/ml, or at least 25 mg/ml.

The term “a mutation that reduces the transport of glucose into the cell” as used herein refers to a mutation in a gene encoding a protein involved in transport of glucose which results in an accumulation of glucose in the environment of the cell.

The level of glucose in the culture medium of a S. thermophilus strain can readily be measured by methods known to the skilled person.

It may be contemplated that the S. thermophilus strain comprises an amino acid sequence which is at least 50% identical to SEQ ID NO: 2, 4, 6, 8, 10 and/or 12. Preferably, the S. thermophilus strain comprises a nucleotide sequence which is at least 60%, at least 70%, at least 80%, at least 90%, at least 95%, at least 96%, at least 97%, at least 98% or at least 99% identical to SEQ ID NO: 2, 4, 6, 8, 10 and/or 12.

In a specific embodiment the mutations on the nucleic acid level are:
(a) a substitution from C to T at a position corresponding to position 169 in SEQ ID NO. 1;
(b) a substitution from G to A at a position corresponding to position 139 in SEQ ID NO. 3;
(c) a substitution from A to C at a position corresponding to position 726 in SEQ ID NO. 5 or 13;
(d) a substitution from C to T at a position corresponding to position 490 in SEQ ID NO. 7;
(e) a substitution from C to T at a position corresponding to position 82 in SEQ ID NO. 9; and
(f) a substitution from G to T at a position corresponding to position 805 in SEQ ID NO: 11.

In a specific embodiment the changes in the encoded protein are:
(a) a substitution of Pro to Leu at a position corresponding to position 57 in SEQ ID NO 2;
(b) a substitution of Ile to Val at a position corresponding to position 47 in SEQ ID NO 4;
(c) a substitution of Glu to Asp at a position corresponding to position 242 in SEQ ID NO 6 or 14;
(d) a substitution of Pro to Ser at a position corresponding to position 164 in SEQ ID NO 8;
(e) a substitution of Leu to Phe at a position corresponding to position 28 in SEQ ID NO 10; and
(f) a substitution of Gly to Cys at a position corresponding to position 268 in SEQ ID NO 12.

In a preferred embodiment the Streptococcus thermophilus strain comprises the following mutations:
(d) a substitution from C to T at a position corresponding to position 169 in SEQ ID NO.1; a substitution from nucleotide G to A at a position corresponding to position 139 in SEQ ID NO. 3 and a substitution from nucleotide A to C at a position corresponding to position 726 in SEQ ID NO. 5 or 13;
(e) a substitution from C to T at a position corresponding to position 490 in SEQ ID NO. 5 or 13 and a substitution from nucleotide C to T at a position corresponding to position 82 in SEQ ID NO. 7; or
(f) a substitution from G to T at a position corresponding to position 805 in SEQ ID NO: 9.

In a preferred embodiment wherein the Streptococcus thermophilus strain comprises the following changes in the encoded protein:
(a) a substitution of Pro to Leu at a position corresponding to position 57 in SEQ ID NO 2; a substitution of Ile to Val at a position 47 in SEQ ID NO 4; and a substitution of Glu to Asp at a position corresponding to position 242 in SEQ ID NO 6 or 14;
(b) a substitution of Pro to Ser at a position corresponding to position 164 in SEQ ID NO 8; and a substitution of Leu to Phe at a position corresponding to position 28 in SEQ ID NO 10; or
(c) a substitution of Gly to Cys at a position corresponding to position 268 in SEQ ID NO 12.

TABLE 2
Strains and specific mutations.

| | Nucleotide | Amino acid | DSM | DSM | DSM |
|---|---|---|---|---|---|
| Gene | substitution | substitution | 33719 | 33720 | 33762 |
| PTS Mannose/ | C169T | Pro57Leu | + | | |
| glucose/fructose | SEQ ID NO. 1 | (P57L) | | | |
| subunit IIC (ManM) | | SEQ ID NO. 2 | | | |
| Galacto kinase | G139A | Ile47Val | + | | |
| (galK) | SEQ ID NO. 3 | (I47V) | | | |
| | | SEQ ID NO. 4 | | | |
| Phosphoglucomutase | A726C | Glu242Asp | + | | |
| (pgm) | SEQ ID NO. 5 | (E242D) | | | |
| | or 13 | SEQ ID NO. 6 | | | |
| | | or 14 | | | |
| Phosphoglucomutase | C490T | Pro164Ser | | + | |
| (pgm) | SEQ ID NO. 7 | (P164S) | | | |
| | | SEQ ID NO. 8 | | | |
| Galactose operon | C82T | Leu28Phe | | + | |
| repressor gene | SEQ ID NO. 9 | (L28F) | | | |
| (galR) | | SEQ ID NO. 10 | | | |
| Glucose kinase | G805T | Gly268Cys | | | + |
| (glcK) | SEQ ID NO. 11 | (G268C) | | | |
| | | SEQ ID NO. 7 | | | |

The identified strains have been deposited. In a preferred embodiment, the invention relates to S. thermophilus strain(s):
(a) DSM 33719 or mutants or variants thereof;
(b) DSM 33720 or mutants or variants thereof; or
(c) DSM 33762 or mutants or variants thereof.

In a preferred embodiment, the invention relates to S. thermophilus strain DSM 33719 or mutants or variants thereof, the S. thermophilus strain DSM 33720 or mutants or variants thereof and/or the S. thermophilus strain 33762 or mutants or variants thereof. In a much-preferred embodiment of the present invention the mutant strain is a naturally occurring mutate or an induced mutant.

To ensure the applicability in industry it may be contemplated that the mutants or variants of DSM 33719 show the same or similar characteristics (such as acidification profile and texturizing properties) as DSM 33719. Likewise, it may be contemplated that the mutants or variants of DSM 33720 show the same or similar characteristics (such as acidification profile and texturizing properties) e.g. as DSM 33720. Similar it may be contemplated that the mutants or variants of DSM 33719 show the same or similar characteristics (e.g. acidification profile) as DSM 33719.

To ensure the applicability in industry it may be contemplated that the mutants or variants of 33719 show the same or similar characteristics (such as acidification profile and texturizing properties) as DSM 33719. Likewise, it may be contemplated that the mutants or variants of 33720 show the same or similar characteristics (such as acidification profile and texturizing properties) e.g. as DSM 33720.

In one embodiment, mutants or variants of DSM 33719 comprises a nucleotide sequence which is at least 50% identical to SEQ ID NO: 1, 3 and/or 5. Preferably, the mutants or variants of DSM 33719 comprises a nucleotide sequence which is at least 60%, at least 70%, at least 80%, at least 90%, at least 95%, at least 96%, at least 97%, at least 98% or at least 99% identical to SEQ ID NO: 1, 3 and/or 5.

In one embodiment, mutants or variants of DSM 33720 the present invention comprises a nucleotide sequence which is at least 50% identical to SEQ ID NO: 7 and/or 9. Preferably, the mutants or variants of DSM 33720 comprises a nucleotide sequence which is at least 60%, at least 70%, at least 80%, at least 90%, at least 95%, at least 96%, at least 97%, at least 98% or at least 99% identical to SEQ ID NO: 7 and/or 9.

In one embodiment, mutants or variants of DSM 33762 comprises a nucleotide sequence which is at least 50% identical to SEQ ID NO: 11. Preferably, the mutants or variants of DSM 33762 comprises a nucleotide sequence which is at least 60%, at least 70%, at least 80%, at least 90%, at least 95%, at least 96%, at least 97%, at least 98% or at least 99% identical to SEQ ID NO: 11.

In one embodiment, mutants or variants of DSM 33719 comprises an amino acid sequence which is at least 50% identical to SEQ ID NO: 1, 3 and/or 5. Preferably, the mutants or variants of DSM 33719 comprises an amino acid sequence which is at least 60%, at least 70%, at least 80%, at least 90%, at least 95%, at least 96%, at least 97%, at least 98% or at least 99% identical to SEQ ID NO: 1, 3 and/or 5.

In one embodiment, mutants or variants of DSM 33720 the present invention comprises an amino acid sequence which is at least 50% identical to SEQ ID NO: 7 and/or 9. Preferably, the mutants or variants of DSM 33720 comprises an amino acid sequence which is at least 60%, at least 70%, at least 80%, at least 90%, at least 95%, at least 96%, at least 97%, at least 98% or at least 99% identical to SEQ ID NO: 7 and/or 9.

In one embodiment, mutants or variants of DSM 33762 comprises an amino acid sequence which is at least 50% identical to SEQ ID NO: 11. Preferably, the mutants or variants of DSM 33762 comprises an amino acid sequence which is at least 60%, at least 70%, at least 80%, at least 90%, at least 95%, at least 96%, at least 97%, at least 98% or at least 99% identical to SEQ ID NO: 11.

As mentioned previously there is a need in the industry to develop new S. thermophilus stains strains with a sweetening property. Thus, in a preferred embodiment the S. thermophilus strain (s) according to according to the present invention are galactose fermenting.

In the present context, the term “galactose fermenting” as defined herein means that pH is reduced by a value of at least 1.0 after 16 hours incubation at 37° C. in M17 with 2% galactose (galactose added as sole carbohydrate), inoculated in an amount of at least 10⁴ cells/ml.

Some strains may be both galactose fermenting and glucose fermenting—this is e.g. the case with DSM 33720 as it can both ferment glucose and galactose. DSM 33720 carries a mutation so that glucose is not or only to a limited extend transported out of the cell. Therefore, glucose is not accumulated in the fermentation product to the same extend as it is with other galactose fermenting strains. DSM 33720 is therefore a strain with a light sweetening property.

Terms such as “strains with a sweetening property”, “strains which can provide a desirable accumulation of glucose in the fermented milk product” and “strains with enhanced properties for natural sweetening of food products” are used interchangeably herein to characterize an advantageous aspect of using the strains of the present invention in fermentation of milk products.

In one embodiment of the present invention the S. thermophilus strain DSM 33720, DMS 33762 and/or DSM 33719 generates a stress greater than 45 Pa, 50 Pa, 60 Pa, 70 Pa, 80 Pa, 90 Pa, or 100 Pa at 300 s⁻¹ when measured on a fermented product made by addition of a composition of DSM 33720, DMS 33762 and/or DSM 33719 and one or more further lactic acid bacteria strain (mixed culture). It may be desired as this resembles a sensory viscosity/mouth thickness which is preferred by a sensory panel. Shear stress is measured as described in Example 4.

It may be contemplated that the S. thermophilus strain(s) of the present invention is/are 2-deoxyglucose resistant. The term “resistant to 2-deoxyglucose” herein in relation to S. thermophilus is defined by that a particular mutated bacterial strain has the ability to grow to a colony when streaked on a plate of M17 medium containing 20 mM 2-deoxyglucose after incubation at 40° C. for 20 hours. The presence of 2-deoxyglucose in the culture medium will prevent the growth of non-mutated strains while the growth of the mutated strains is not affected or not affected significantly. Thus, for selection purposes 2-deoxyglucose can be applied in the selection process.

In an embodiment the S. thermophilus strain DSM 33720 carries mutations that make the strain hyper-galactose fermenting.

It will be appreciated that aspects and embodiments disclosed in the parts termed “Composition” and “Method of producing a fermented product” may be equally relevant to the present part of the specification.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the fat encapsulated microbial culture and all its features, which may readily be part of the final composition or product obtained by the method as described herein. Embodiments and features of the present invention are also outlined in the following items.

### Deposits and Expert Solutions

The applicant requests that a sample of the deposited microorganisms stated in the table below may only be made available to an expert, until the date on which the patent is granted.

TABLE 3
Deposits made at a Depositary institution having acquired the
status of international depositary authority under the Budapest
Treaty on the International Recognition of the Deposit of Microorganisms
for the Purposes of Patent Procedure: Leibniz Institute DSMZ-
German Collection of Microorganisms and Cell Cultures Inhoffenstr.
7B, 38124 Braunschweig, Germany.

| Strain | Accession No. | Deposit date |
|---|---|---|
| Streptococcus thermophilus | DSM 33719 | 2020 Nov. 26 |
| Streptococcus thermophilus | DSM 33762 | 2021 Jan. 19 |
| Streptococcus thermophilus | DSM 33720 | 2020 Nov. 26 |

### EXAMPLES

### Materials and Methods

TABLE 4
Medium compositions with amount of ingredients per liter H₂0

| M17 agar medium: | M17 broth: |
|---|---|
| agar, 12.75 g | ascorbic acid, 0.5 g |
| ascorbic acid, 0.5 g | magnesium sulfate, 0.25 g |
| casein peptone (tryptic), 2.5 g | meat extract, 5 g |
| disodium β-glycerophosphate penta | meat peptone (peptic), 2.5 g |
| hydrate, 19 g | sodium glycerophosphate, 19 g |
| magnesium sulfate hydrate, 0.25 g | soya peptone (papainic), 5 g |
| meat extract, 5 g | tryptone, 2.5 g |
| meat peptone (peptic), 2.5 g | yeast extract, 2.5 g |
| soyapeptone (papainic), 5 g | final pH 7.0 ± 0.2 (25° C.) |
| yeast extract, 2.5 g | |
| final pH 7.1 ± 0.2 (25° C.) | |

Carbon sources added sterile:
lactose 20 g/l
glucose 20 g/l, 25 g/l or 50 g/l
galactose 20 g/l, 25 g/l or 50 g/l.

As known in the art M17 medium is considered suitable for growth of S. thermophilus. The skilled person, in the present context, understands that a M17 concentrate may be supplied from different suppliers and independently of the specific supplier one will (within standard measurement uncertainty) get the same herein relevant result of 2-deoxyglucose resistance for a herein relevant cell of interest.

Streptococcus thermophilus strains DSM 33762 is a 2-deoxyglucose resistant mutant and DSM 33720 is a hyper-galactose fermenting strain both of which were derived from the same galactose fermenting mother strain (MS-1).

DSM 33719 is a fast glucose secreting strain derived from a hyper-lactose fermenting and glucose secreting mother strain (MS-2).

### Milk Acidification Profiles and Carbohydrate Analysis

B-milk was used as substrate. B-milk consists of skim milk powder at a level of dry matter of 9.5% (w/v) reconstituted in distilled water and pasteurized at 99° C. for 30 min, followed by cooling to 30° C.

Acidification was followed using a CINAC prior art hardware and software or an ICINAC system (AMS alliance). This system is developed to monitor the acidification activity of lactic ferments and can simultaneously follow and register the change in pH and temperature using electrodes.

The content of carbohydrates was investigated by using HPLC analysis. Aliquots of acidified B-milk were collected after approximately 20 hours of acidification at 40° C. The amount of carbohydrates is presented in g/l.

### EXAMPLE 1—Use of 2-deoxyglucose to Isolate Glucose Kinase Mutants of Streptococcus thermophilus with Enhanced Excretion of Glucose

In order to isolate mutants MS-1 cells derived from the growth of a single colony were inoculated into 10 ml of M17 broth containing 2% lactose and grown overnight at 40° C.

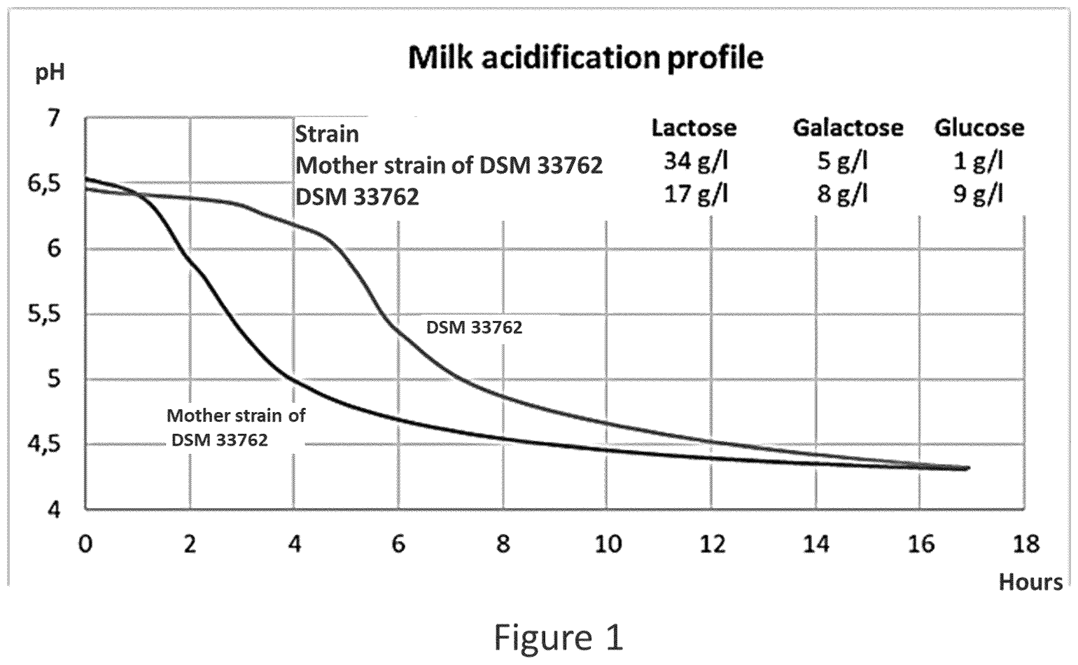

Next day, the culture was plated in serial dilutions on M17 agar plates containing 2% galactose and 30 mM 2-deoxyglucose and incubated for 20 hours at 40° C. Resistant colonies were at first re-streaked on the same type of agar plates used for selection. Survivors were used to inoculate fresh M17 broth containing either 2% lactose, 2% galactose or 2% glucose and growth was measured. Among the selected mutants DSM 33762 was identified. This isolate secreted glucose when grown in milk (FIG. 1) and genome sequencing led to the identification of a mutation in the gene encoding glucokinase.

From FIG. 1 it is apparent that DSM 33762 has a slower acidification profile as compared to MS-1. Furthermore, carbohydrate analysis of DSM 33762 shows that the content of lactose is reduced, whereas both galactose and glucose secretion is increased as compared to a fermented milk prepared by MS-1.

### EXAMPLE 2—Use of Adaptive Laboratory Evolution to Isolate Mutants of Streptococcus thermophilus with a Faster Growth Rate on Galactose Containing Medium

Adaptive laboratory evolution (ALE) was used to generate galactose fermenting mutants with a faster growth rate in media with galactose as sole sugar source as compared to their mother strain.

ALE was conducted with 3 parallel cultures of MS-1 which were grown for 4 weeks in M17 medium with 5% galactose+2% casein hydrolysate. ALE was performed according to a protocol essentially as described in Troy E. Sandberg, Michael J. Salazar, Liam L. Weng, Bernhard O. Palsson, Adam M. Feist. The emergence of adaptive laboratory evolution as an efficient tool for biological discovery and industrial biotechnology, Metabolic Engineering, Volume 56, 2019, Pages 1-16 (Sandberg et al. 2019).

The adapted cultures were tested every week for growth rate improvements. This was performed by testing an aliquot of the adapted cultures directly in the same medium used for ALE process and then also by plating dilutions of the adapted cultures on M17 agar plates containing galactose. Single colonies from these plates were purified and then tested for optimized performance in the M17 medium with galactose+casein hydrolysate. The majority of the isolates showed a significant improvement in growth rate over MS-1. DSM 33720 was isolated after 3 weeks of ALE.

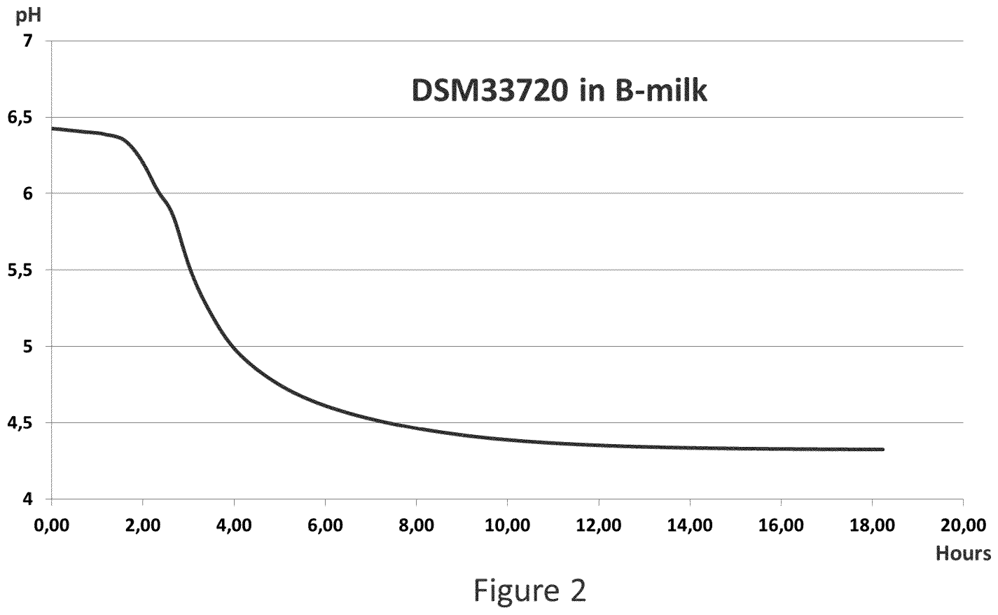

In milk, DSM 33720 was found both to be very texturizing and to have a fast acidification rate (FIG. 2).

EXAMPLE 3—Use of Adaptive Laboratory Evolution to Isolate Glucose Secreting Mutants of S. thermophilus with a Faster Growth Rate on Galactose and Lactose Containing Medium

MS-2 is a strain with high glucose secretion due to double mutation (glcK, Glu/Man PTS) as described in WO2013/160413. However, such mutants often show slower performance in milk and it was therefore an aim to isolate faster derivatives using ALE. 3 parallel cultures of MS-2 were grown for 3 weeks in M17 medium with 5% galactose +2% casein hydrolysates. In week 4 the medium was changed to M17 with 2.5% galactose+2.5% lactose+2% casein hydrolysates. ALE was performed according to a protocol essentially as described in Sandberg et al. 2019.

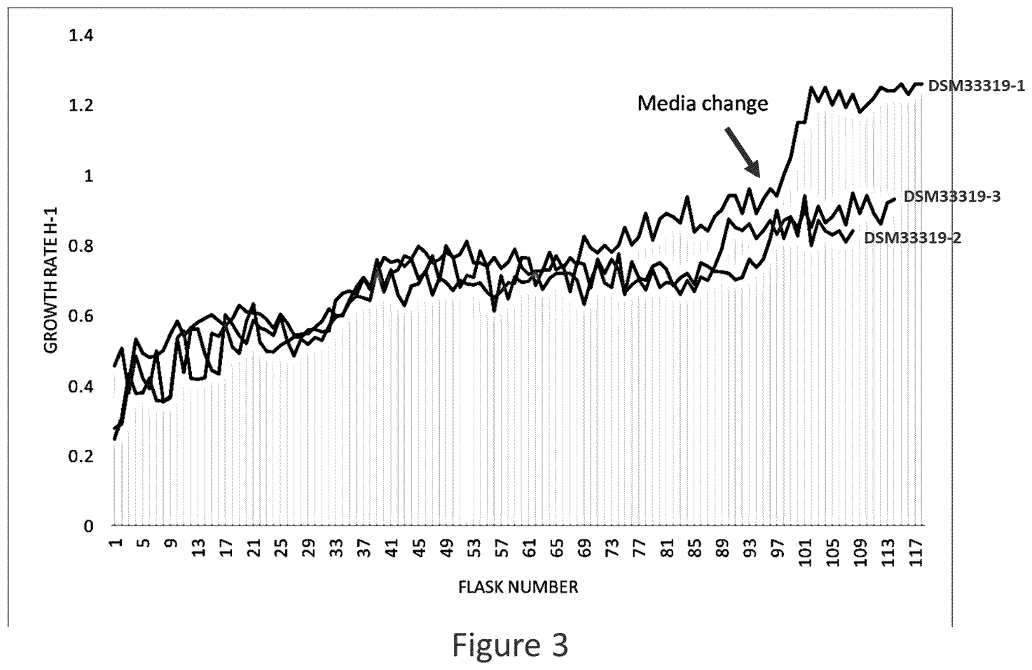

The graph in FIG. 3 represents the entire 4 weeks of growth where the cultures daily or several times daily were diluted and regrown until faster growth were achieved. Starting from a low base of growth rate, the growth rate increase was significant after 4 weeks. After 4 weeks, a diluted culture was plated from each 3 parallel cultures and picker single colonies from these agar plating's. DSM 33719 is one such isolate coming from the grey curve (culture 3) in FIG. 3.

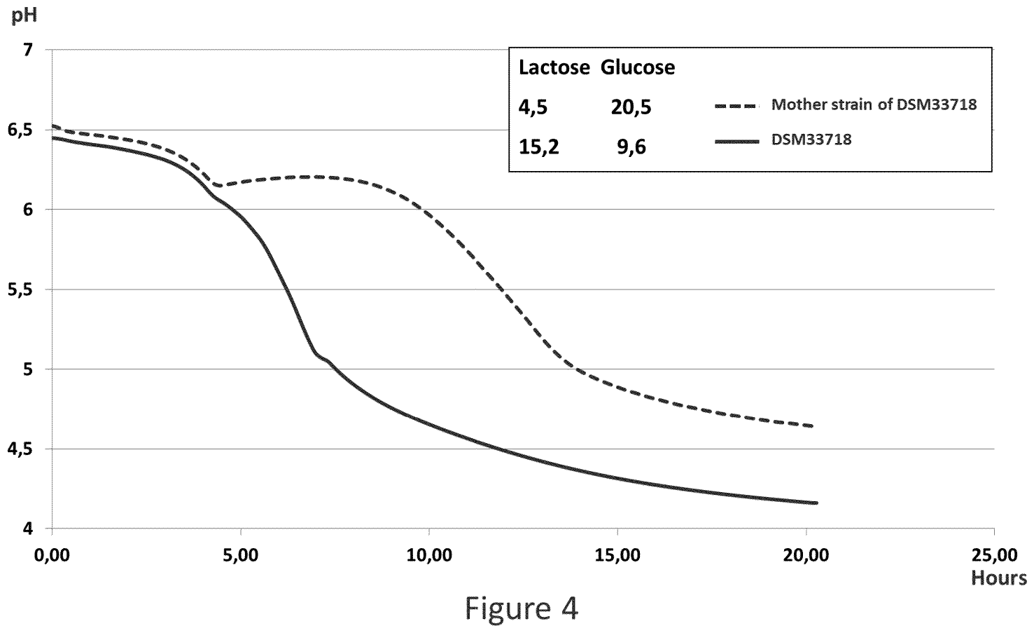

FIG. 4 shows the results of DSM 33719. The acidification with DSM 33719 was much faster as compared to MS-2 and DSM 33719 acidified to a lower pH. Carbohydrate analysis of the fermented milk gave an indication why DSM 33719 was faster. The glucose secretion was reduced to half and it was also observed that DSM 33719 adapted well to grow on peptides (casein hydrolysate). Further analysis of DSM 33719 showed that in addition to showing faster acidification especially with casein hydrolysate added or in pre-culture, it was also found that DSM 33719 provided improved texture to milk if part of a mixed culture (see Example 4).

### EXAMPLE 4—Milk Acidification and Carbohydrate Analysis of Milk Fermented with Different Combinations and Ratios of Strains

TABLE 5
Culture strain combination

| | DSM | DSM | DSM | DSM | DSM | DSM | DSM | DSM |
|---|---|---|---|---|---|---|---|---|
| | 28889 | 26722 | 25850 | 33720 | 33719 | 32227 | 33762 | 28910 |
| Culture 1 | x | x | x | | | | | x |
| Culture 2 | | | | | | x | x | x |
| Culture 3 | x | x | x | x | | | | x |
| Culture 4 | | | | | x | x | | x |

The yogurt cultures Premium 1.0 and YF-L904 sold by Chr. Hansen were included as reference.

Two different milk bases were produced prior to fermentation. Skimmed milk powder and sucrose were added to the milk with subsequent 2-hour hydration without stirring, where after homogenization and pasteurization for 1 minute at >=80° C. was conducted.

Milk base 1 contained 0.1% sucrose, 4% protein (adjusted with skimmed milk powder) and 1.5% fat (1.5% fat milk adjusted with 9% cream). Milk base 2 contained 5% sucrose, 4% protein (adjusted with skimmed milk powder) and 1.5% fat (1.5% fat milk adjusted with 9% cream).

The milk bases were transferred into 200 mL baby bottles. The bottles were inoculated with a 100× liquid dilution prepared from frozen pellets (F-DVS) dissolved in B-milk. For each strain, an individual dilution was prepared. In order to create a blend, several strains were inoculated into 1 CINAC bottle in differing ratios and volumes. The final inoculation rate of the blends corresponded 1E+6−1E+8 CFU/mL.

Apart from DSM 33762, which was used in Culture 2, all strains were inoculated from a first dilution created from F-DVS. For preparation of the DSM 33762 inoculation material, 1 mL M-17 B-K medium with 4% sucrose and casein peptone in excess was inoculated with a scrap of DSM 33762 and incubated at 43° for at least 18 hours.

The inoculated bottles were placed into a water bath and heated to 43° C.

pH was monitored at 43° C. according to prior art using CINAC hardware and software until the blends reached the final pH of 4.55. In order to achieve a homogenous gel of the fermented milk, each bottle was stirred and treated in a Micro Application Platform (MAP) at 25° C. and 2 bar. This MAP is a miniature, lab-based equipment equal the prior-art Post-treatment Unit (PTU) used in dairies.

Fermented milk samples were prepared for carbohydrate analysis using HPLC by weighing 1 g+/−0.5 g into 10 mL centrifuge tubes. 2 mL ice cold 96% ethanol was added, the samples where whirly-mixed and placed at −50° C. until analyzed by HPLC.

The baby bottles with the fermented milk samples were stored at 5° C. for 7 days before texture analysis (rheology). For rheological measurement, the samples were transferred into bob cups and analyzed for Shear Stress.

Shear stress was measured by using ASC rheometer model DSR502 from Anton Paar. The method is using a rotational step which is based on a rotational deformation on the sample, from 10-3 s−1 to 300 s−1, and then back to 10-3 s−1. The corresponding shear stress is measured. For these results, one shear rate (300 s−1) was extracted from the flow curve. Samples were placed at 13° C. for 1 hour prior to measuring. Each sample was gently stirred with a spoon 5 times from bottom to top to assure a homogenous sample. The rheology cups were filled until the line and placed in the sample magazine. Samples were measured in duplicates using two separate yogurt cups. Measurements were conducted at day +7 and temperature of measurement is set to 13° C. Samples were stored at 5° C. until the day of measurement.

TABLE 6
Fermentation time, texture and glucose levels obtained
with Culture 1, Culture 3 and Culture 4.

| | Fermentation time to | Shear Stress | |
|---|---|---|---|
| | target pH 4.55 | at 1*300 | Glucose |
| Milk base 1 | (hours) | (Pa) | (mg/g) |
| Premium 1.0 | 5.5 | 55 | 0 |
| YF-L904 | 5.4 | 55 | 0 |
| Culture 1 | 11.1 | 30 | 18.7 |
| Culture 3 (Blend 1-8) | 7.8-8.6 | 50-55 | 5-8 |
| Culture 4 (Blend 1-6) | 9-12 | 55-60 | 9-15 |

The trial was conducted in milk base 1. The results for Culture 3 and Culture 4 are obtained from different blends, i.e. e.g. each of the 8 Culture 3 blends comprise the same strains but in a different amount and ratios.

The fermentation time, texture and glucose levels are dependent on the culture/blend. Culture 3 and Culture 4 provide equally good texture as one of the best texturing cultures Premium 1.0, whereas Culture 1 provides much lower texture in the fermented milk product. Different ratios of strains in the blend variants can lead to variation in i.e. fermentation time, texture or glucose levels. Thus, the composition of a culture and a blend can accordingly be adjusted to address the specific needs required in the final fermented product.

TABLE 7
Fermentation time, texture and glucose levels
obtained with Culture 1 and Culture 2.

| | Fermentation time | Shear Stress | Concentration |
|---|---|---|---|
| | to target pH 4.55 | at 1*300 | of Glucose |
| | (hours) | (Pa) | (mg/g) |
| Milk Base 1: | | | |
| Culture 1 | 12 | 25 | 16 |
| Culture 2 (Blend 1-6) | 8-11 | 20-30 | 5-6 |
| Milk base 2: | | | |
| Culture 1 | 14 | 27 | 16 |
| Culture 2 (Blend 1-6) | 8-10 | 25-30 | 4-5 |

The table above shows results obtained with Culture 1 and Culture 2 (Blend 1-6). Blend 1-6 are six blend variations comprising the same strains but in different amounts and ratios. It can be seen that Culture 2 results in similar texture in both milk bases compared to Culture 1. At the same time, Culture 2 results in shorter fermentation time and lower glucose levels.

### EXAMPLE 5—Altered Ratio of Strains in a Blend Changes Properties of the Fermented Milk Product

Fermented milk was produced and analyzed as described in Example 4.

TABLE 8
Composition of Culture 4 blend variants (% of Total)

| | Blend variant S03b | Blend variant S03 | Blend variant P07 |
|---|---|---|---|
| DSM 33719 | 5.5 | 11.0 | 17.5 |
| DSM 33720 | 89.0 | 83.5 | 78.0 |
| DSM 28910 | 5.5 | 5.5 | 4.5 |

TABLE 9
Fermentation time, texture and glucose levels obtained
with Culture 4 blend variants in Milk base 1

| | Fermentation time | Shear Stress | Concentration |
|---|---|---|---|
| | to target pH 4.60 | at 1*300 | of Glucose |
| Milk base 1 | (hours) | (Pa) | (mg/g) |
| Culture 1 | 11.0 | 26 | 19.7 |
| Culture 4 - S03b | 11.5 | 60 | 14.5 |
| Culture 4 - S03 | 10.7 | 64 | 12.9 |
| Culture 4 - P07 | 10.2 | 64 | 12.7 |

TABLE 10
Fermentation time, texture and glucose levels obtained
with Culture 4 blend variants in Milk base 2.

| | Fermentation time | Shear Stress | Concentration |
|---|---|---|---|
| | to target pH 4.60 | at 1*300 | of Glucose |
| Milk base 2 | (hours) | (Pa) | (mg/g) |
| Culture 1 | 12.6 | 29 | 17.3 |
| Culture 4 - S03b | 12.0 | 55 | 8.8 |
| Culture 4 - S03 | 9.0 | 54 | 5.9 |
| Culture 4 - P07 | 8.6 | 55 | 5.3 |

Different ratios of strains in the blend variants of Culture 4 (S03b, S03 and P07) lead to variation in fermentation times, texture (Shear stress at 300 1*s) and glucose levels. In both Milk base 1 and Milk base 2 the blend variants are superior in texture as compared to Culture 1. In milk base 2, Culture 4 blend variants showed a faster fermentation as compared to Culture 1. Thus, the characteristic of a Culture/blend is determined by the properties of the individual strain and the amount of the strain present leading to different fermented products. The composition of a culture/blend can accordingly be adjusted to address specific needs in the final fermented product.

### Items

Item Y1. A composition comprising a Streptococcus thermophilus strain having a mutation in one or more genes selected from the group consisting of:
   (a) the manM gene encoding the PTS Mannose/glucose/fructose subunit IIC at a position corresponding to position 169 in SEQ ID NO. 1;
   (b) the galK gene encoding the galactokinase at a position corresponding to position 139 in SEQ ID NO. 3;
   (c) the pgm gene encoding the phosphoglucomutase at a position corresponding to position 490 in SEQ ID NO. 5 or 13;
   (d) the pgm gene encoding the phosphoglucomutase at a position corresponding to position 726 in SEQ ID NO. 7;
   (e) the galR gene encoding the galactose operon repressor at a position corresponding to position 82 in SEQ ID NO. 9; and
   (f) the glcK gene encoding the glucose kinase at a position corresponding to position 805 in SEQ ID NO. 11.
Item Y2. The composition according to item Y1, wherein the mutation leads to a change in the encoded protein selected from the group consisting of:
   a) the PTS Mannose/glucose/fructose subunit IIC at a position corresponding to position 57 in SEQ ID NO 2;
   b) the galactokinase at a position corresponding to position 47 in SEQ ID NO 4;
   c) the phosphoglucomutase at a position corresponding to position 242 in SEQ ID NO 6 or 14;
   d) the phosphoglucomutase at a position corresponding to position 164 in SEQ ID NO 8;
   e) the galactose operon repressor at a position corresponding to position 28 in SEQ ID NO 10; and
   f) the glucose kinase at a position corresponding to position 268 in SEQ ID NO 12.
Item Y3. The composition according to any one of the preceding items, wherein the mutations are:
   (a) a substitution from C to T at a position corresponding to position 169 in SEQ ID NO. 1;
   (b) a substitution from G to A at a position corresponding to position 139 in SEQ ID NO. 3;
   (c) a substitution from A to C at a position corresponding to position 726 in SEQ ID NO. 5 or 13;
   (d) a substitution from C to T at a position corresponding to position 490 in SEQ ID NO. 7;
   (e) a substitution from C to T at a position corresponding to position 82 in SEQ ID NO. 9; and
   (f) a substitution from G to T at a position corresponding to position 805 in SEQ ID NO: 11.
Item Y4. The composition according to any one of the preceding items, wherein the changes in the encoded protein are:
   (a) a substitution of Pro to Leu at a position corresponding to position 57 in SEQ ID NO 2;
   (b) a substitution of Ile to Val at a position corresponding to position 47 in SEQ ID NO 4;
   (c) a substitution of Glu to Asp at a position corresponding to position 242 in SEQ ID NO 6 or 14;
   (d) a substitution of Pro to Ser at a position corresponding to position 164 in SEQ ID NO 8;
   (e) a substitution of Leu to Phe at a position corresponding to position 28 in SEQ ID NO 10; and
   (f) a substitution of Gly to Cys at a position corresponding to position 268 in SEQ ID NO 12.
Item Y5. The composition according to any one of the preceding items, wherein the Streptococcus thermophilus strain comprises the following mutations:
   (a) a substitution from C to T at a position corresponding to position 169 in SEQ ID NO.1; a substitution from nucleotide G to A at a position corresponding to position 139 in SEQ ID NO. 3 and a substitution from nucleotide A to C at a position corresponding to position 726 in SEQ ID NO. 5 or 13;
   (b) a substitution from C to T at a position corresponding to position 490 in SEQ ID NO. 5 or 13 and a substitution from nucleotide C to T at a position corresponding to position 82 in SEQ ID NO. 7; or
   (c) a substitution from G to T at a position corresponding to position 805 in SEQ ID NO: 9.
Item Y6. The composition according to any one of the preceding items, wherein the Streptococcus thermophilus strain comprises the following changes in the encoded protein:
   (a) a substitution of Pro to Leu at a position corresponding to position 57 in SEQ ID NO 2; a substitution of Ile to Val at a position 47 in SEQ ID NO 4; and a substitution of Glu to Asp at a position corresponding to position 242 in SEQ ID NO 6 or 14;
   (b) a substitution of Pro to Ser at a position corresponding to position 164 in SEQ ID NO 8; and a substitution of Leu to Phe at a position corresponding to position 28 in SEQ ID NO 10; or
   (c) a substitution of Gly to Cys at a position corresponding to position 268 in SEQ ID NO 12.
Item Y7. The composition according to any one of the preceding items, wherein the Streptococcus thermophilus strain is:
   (a) DSM 33719 or mutants or variants thereof;
   (b) DSM 33720 or mutants or variants thereof; or
   (c) DSM 33762 or mutants or variants thereof.
Item Y8. The composition according to any one of the preceding items, wherein the composition comprises one or more Streptococcus thermophilus strains selected from the group comprising: DSM 33719, DSM 33719 mutants, DSM 33719 variants, DSM 33720, DSM 33720 mutants, DSM 33720 variants, DSM 33762, DSM 33762 mutants, and DSM 33762 variants.
Item Y9. The composition according to the preceding item, wherein the composition comprises:
   (a) DSM 33720, DSM 33719, and DSM 33762;
   (b) DSM 32227 and DSM 33762; or
   (c) DSM 33719 and DSM 32227.
Item Y10. The composition to any one of the preceding items, further comprising one or more strains belonging to the genus Lactobacillus.
Item Y11. The composition according to item Y10, wherein the one or more strains of Lactobacillus is selected from a group consisting of: Lactobacillus delbrueckii, Lactobacillus delbrueckii subsp bulgaricus, Lactobacillus delbrueckii subsp. lactis, Lactobacillus acidophilus, Lacticaseibacillus casei Lacticaseibacillus paracasei subsp. Paracasei, and Lacticaseibacillus rhamnosus, Limosilactobacillus fermentum, Lactiplantibacillus plantarum subsp. Plantarum and Lactobacillus helveticus.
Item Y12. The composition according to any one of Y9-Y10, wherein the Lactobacillus delbrueckii subsp bulgaricus is DSM 28910 or mutants or variants thereof
Item Y13. The composition according to any one of the preceding items comprising, either as a mixture or as a kit-of-parts:
   (a) a S. thermophilus strain selected from the group consisting of DSM 33720 or mutants or variants thereof, DSM 33719 or mutants or variants thereof, DSM 33762 or mutants or variants thereof, or any combination thereof; and
   (b) a strain belonging to the genus Lactobacillus.
Item Y14. The composition according to any of the preceding items, wherein the composition is a starter culture.
Item Y15. The composition according to any of the preceding claims, wherein the composition is in a frozen, spray-dried, freeze-dried, vacuum-dried, air dried, tray dried or liquid form.
Item Z1. A method of producing a fermented product, comprising fermenting a substrate with a Streptococcus thermophilus strain having mutations in one or more genes selected from the group consisting of (a)-(f) of item 1.
Item Z2. The method according to item Z1, wherein the substrate is a milk substrate.
Item Z3. The method according to any one of items Z1-Z2, wherein the milk substrate is derived from an animal.
Item Z4. The method according to any one of items Z1-Z3, wherein the milk substrate further comprises a plant derived substrate.
Item Z5. The method according to any one of items Z1-Z4, wherein the fermented product is a food product.
Item Z6. The method according to any one of items Z1-Z3, wherein the food product is a dairy product.
Item Z7. The method according to item Z6, wherein the dairy product is selected from the group consisting of a fermented milk product (e.g. yoghurt, buttermilk or kefir) or a cheese (e.g. fresh cheese or pasta filata)
Item Z8. The method according to any one of items Z1-Z7, wherein the fermented product further comprises an ingredient selected from the group consisting of a fruit concentrate, a syrup, a probiotic bacterial strain or culture, a coloring agent, a thickening agent, a flavoring agent, a preserving agent and mixtures thereof.
Item Z9. The method according to item Z8, wherein an enzyme is added to the substrate before, during and/or after the fermenting, the enzyme being selected from the group consisting of an enzyme able to crosslink proteins, transglutaminase, an aspartic protease, chymosin, rennet, lactase and mixtures thereof.
Item Z10. The method according to any one of items Z1-Z9, wherein the fermented product is in the form of a stirred type product, a set type product, or a drinkable product.
Item Q1. A fermented product obtainable by the method according to any one of items Z1-Z10.
Item Q2. The fermented product according to item Q1, wherein the fermented product is a food product.
Item Q3. The fermented product according to any one of items Q1-Q2 wherein the food product is a dairy product.
Item P1. A fermented product comprising one or more Streptococcus thermophilus strain having a mutation in one or more genes selected from the group consisting of (a)-(f) of item Y1.
Item P2. The fermented product according to item P1, wherein the fermented product is a food product.
Item P3. The fermented product according to item P1, wherein the food product is a dairy product.
Item W1. Use of one or more S. thermophilus strain having a mutation in one or more genes selected from the group consisting of (a)-(f) of item Y1 for the manufacture of a fermented product.
Item W2. The use according to W1, wherein the fermented product is a food product.
Item W3. The use according to any one of W1-W2, wherein the food product is a dairy product.
Item X1. A Streptococcus thermophilus strain having a mutation in one or more genes selected from the group consisting of (a)-(f) of item Y1.
Item X3. The Streptococcus thermophilus strain according to any one of items X1-X2, wherein the S. thermophilus strain is galactose fermenting.
Item X4. The Streptococcus thermophilus strain according any one of items X1-X3, wherein the S. thermophilus strain is 2-deoxyglucose resistant.
Item X5. The Streptococcus thermophilus strain according any one of items X1-X4, wherein the S. thermophilus carries a mutation that reduces the transport of glucose into the cell.
6. The Streptococcus thermophilus strain according any one of items X1-X5, wherein the S. thermophilus increases the amount of glucose in 9.5% B-milk to at least 5 mg/ml when inoculated into the 9.5% B-milk at a concentration of 1.0E06-1.0E07 CFU/ml and grown at 40° C. for 20 hours.
Item X7. The Streptococcus thermophilus strain according any one of items X1-X6, wherein the S. thermophilus strain increases the amount of glucose in 9.5% B-milk with 0.05% sucrose to at least 5 mg/ml when inoculated into the 9.5% B-milk with 0.05% sucrose at a concentration of 1.0E06-1.0E07 CFU/ml and grown at 40° C. for 20 hours.

### Sequence Listing

SEQ ID NO 1: ManM gene encoding the PTS Mannose/glucose/fructose subunit IIC—Nucleotide sequence
SEQ ID NO 2: PTS Mannose/glucose/fructose subunit IIC (ManM)—Amino Acid sequence
SEQ ID NO 3: galK encoding the galactokinase—Nucleotide sequence
SEQ ID NO 4: Galactokinase (GalK)—Amino Acid sequence
SEQ ID NO 5: pgm encoding the phosphoglucomutase—Nucleotide sequence
SEQ ID NO 6: Phosphoglucomutase (Pgm)—Amino Acid sequence
SEQ ID NO 7: pgm encoding the phosphoglucomutase—Nucleotide sequence
SEQ ID NO 8: Phosphoglucomutase (Pgm)—Amino Acid sequence
SEQ ID NO 9: gal encoding the galactose operon repressor—Nucleotide sequence
SEQ ID NO 10: Galactose operon repressor (GalR)—Amino Acid sequence
SEQ ID NO 11: glcK encoding the glucose kinase—Nucleotide sequence
SEQ ID NO 12: Glucose kinase (GlcK)—Amino Acid sequence
SEQ ID NO 13: pgm encoding the phosphoglucomutase—Nucleotide sequence
SEQ ID NO 14: Phosphoglucomutase (Pgm)—Amino Acid sequence

1. A composition comprising a Streptococcus thermophilus strain having a mutation that leads to one or more changes in an encoded protein selected from:
(a) a PTS Mannose/glucose/fructose subunit IIC at a position corresponding to position 57 in SEQ ID NO 2;
(b) a galactokinase at a position corresponding to position 47 in SEQ ID NO 4;
(c) a phosphoglucomutase at a position corresponding to position 242 in SEQ ID NO 6 or SEQ ID NO 14;
(d) a phosphoglucomutase at a position corresponding to position 164 in SEQ ID NO 8;
a galactose operon repressor at a position corresponding to position 28 in SEQ ID NO 10; and
a glucose kinase at a position corresponding to position 268 in SEQ ID NO 12.

2. The composition according to claim 2, wherein the one or more changes in the encoded protein are respectively selected from:
(a) a substitution of Pro to Leu at a position corresponding to position 57 in SEQ ID NO 2;
(b) a substitution of Ile to Val at a position corresponding to position 47 in SEQ ID NO 4;
(c) a substitution of Glu to Asp at a position corresponding to position 242 in SEQ ID NO 6 or SEQ ID NO 14;
(d) a substitution of Pro to Ser at a position corresponding to position 164 in SEQ ID NO 8;
(e) a substitution of Leu to Phe at a position corresponding to position 28 in SEQ ID NO 10; and
(f) a substitution of Gly to Cys at a position corresponding to position 268 in SEQ ID NO 12.

3. The composition according to claim 1, wherein the Streptococcus thermophilus strain is selected from:
(a) strain DSM 33719 deposited at Leibniz Institute DSMZ-German Collection of Microorganisms and Cell Cultures (Braunschweig, Germany (DSMZ), and mutants and variants thereof;
(b) strain DSM 33720 deposited at DSMZ, and mutants and variants thereof; and
(c) strain DSM 33762 deposited at DSMZ, and mutants and variants thereof.

4. The composition according to claim 1, wherein the composition comprises strains selected from:
(a) strains deposited at DSMZ under accession numbers DSM 33720, DSM 33719, and DSM 33762;
(b) strains deposited at DSMZ under accession numbers DSM 32227 and DSM 33762; and
(c) strains deposited at DSMZ under accession numbers DSM 33719 and DSM 32227.

5. The composition according claim 1, further comprising one or more strains belonging to genus Lactobacillus.

6. A composition or kit-of-parts comprising:
(a) a Streptococcus thermophilus strain selected from strain DSM 33720 and mutants and variants thereof, strain DSM 33719 and mutants and variants thereof, strain DSM 33762 and mutants and variants thereof, and any combination thereof; and
(b) a strain belonging to genus Lactobacillus.

7. A method of producing a fermented product, comprising fermenting a substrate with the Streptococcus thermophilus strain of claim 12.

8. The method according to claim 7, wherein the substrate is a milk substrate.

9. A fermented product obtained by the method according to claim 7.

10. A fermented product comprising one or more Streptococcus thermophilus strains of claim 12.

11. (canceled)

12. A Streptococcus thermophilus strain having one or more mutations that leads to a change one or more changes in an encoded protein selected from:
(a) a PTS Mannose/glucose/fructose subunit IIC at a position corresponding to position 57 in SEQ ID NO 2;
(b) a galactokinase at a position corresponding to position 47 in SEQ ID NO 4;
(c) a phosphoglucomutase at a position corresponding to position 242 in SEQ ID NO 6 or SEQ ID NO 14;
(d) a phosphoglucomutase at a position corresponding to position 164 in SEQ ID NO 8; 10: and
(e) a galactose operon repressor at a position corresponding to position 28 in SEQ ID NO
(f) a glucose kinase at a position corresponding to position 268 in SEQ ID NO 12.

13. The Streptococcus thermophilus strain according to claim 12, wherein the one or more changes in the encoded protein are respectively selected from:
(a) a substitution of Pro to Leu at a position corresponding to position 57 in SEQ ID NO 2;
(b) a substitution of Ile to Val at a position corresponding to position 47 in SEQ ID NO 4;
(c) a substitution of Glu to Asp at a position corresponding to position 242 in SEQ ID NO 6 or SEQ ID NO 14;
(d) a substitution of Pro to Ser at a position corresponding to position 164 in SEQ ID NO 8;
(e) a substitution of Leu to Phe at a position corresponding to position 28 in SEQ ID NO 10; and
(f) a substitution of Gly to Cys at a position corresponding to position 268 in SEQ ID NO 12.

14. The Streptococcus thermophilus strain according to claim 12, wherein the changes in the encoded protein are selected from:
(a) a substitution of Pro to Leu at a position corresponding to position 57 in SEQ ID NO 2; a substitution of Ile to Val at a position corresponding to position 47 in SEQ ID NO 4; and a substitution of Glu to Asp at a position corresponding to position 242 in SEQ ID NO 6 or SEQ ID NO 14;
(b) a substitution of Pro to Ser at a position corresponding to position 164 in SEQ ID NO 8; and a substitution of Leu to Phe at a position corresponding to position 28 in SEQ ID NO 10; and
(c) a substitution of Gly to Cys at a position corresponding to position 268 in SEQ ID NO 12.

15. The Streptococcus thermophilus strain according to claim 12, wherein the Streptococcus thermophilus strain is selected from:
(a) strain DSM 33719 and mutants and variants thereof;
(b) strain DSM 33720 and mutants and variants thereof; and
(c) strain DSM 33762 and mutants and variants thereof.
